(19) United States
(12) Patent Application Publication
(10) Pub. No.: US 2005/0107611 A1
(43) Pub. Date: May 19, 2005

(54) Pyrazolyl-substituted triazoloquinoxalines

(76) Inventors: Barbara Matuszczak, Innsbruck (AT); Christa E. Muller, Bonn (DE)

Correspondence Address: YOUNG & THOMPSON, 745 SOUTH 23RD STREET, 2ND FLOOR, ARLINGTON, VA, 22202, US

(21) Appl. No.: 10/499,146

(22) PCT Filed: Dec. 19, 2002
(86) PCT No.: PCT/AT02/00361

(30) Foreign Application Priority Data
Dec. 21, 2001 (DE) A2025/2001

(51) Int. Cl. C07D 487/14

(52) U.S. Cl. 544/348

(57) ABSTRACT

The invention relates to derivatives of the pyrazoyl[1,2,4]triazolo[4,3-α]quinoxaline according to formula (1), or of its analog in the form of the pyrazolyl-substituted tetrazolo[1,5-α]quinoxalines according to formula (2). The substances according to formula 1 or 2, in this form or in the form of their pharmaceutically acceptable salts, are suitable as active agents, especially as adenosine receptor ligands.

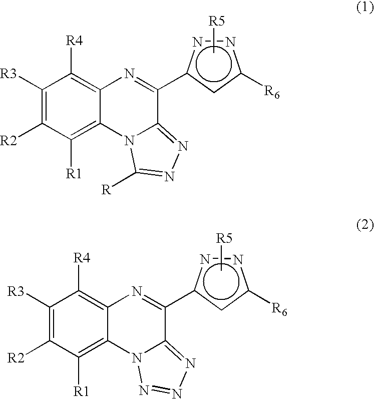

The invention relates to pyrazolyl-substituted triazoloquinoxalines, their analogous tetrazoloquinoxalines as well as their pharmaceutically compatible salts, their production as well as their use as pharmaceutical agents for treating diseases in the kidney area (as K⁺-saving diuretic agents, for acute renal failure, for nephritis, for hepatorenal syndrome); for treating the heart (preferably in the case of cardiac irregularities, ischemia, myocardial infarction or angina pectoris); the central nervous system (for dementia, Alzheimer's disease, anxiety disorders, epilepsy, Parkinson's disease, stroke, depression, opiate withdrawal and comatose conditions); and for treating the lungs (for therapy of respiratory diseases, for example asthma, bronchitis and mucoviscidosis as well as protective agents for a lung transplant). In addition, the invention comprises pharmaceutical agents for treating hypertension, allergic skin diseases (urticaria), inflammations, as immunostimulants, for reducing sperm motility, from which a contraceptive action is derived, as well as diagnostic agents.

### PRIOR ART

It is known that the nucleoside adenosine is a modulator that is ubiquitous in mammals, including humans, and said modulator is bonded extremely tightly to the energy balance in the form of its di- and triphosphates. Adenosine itself has an effect on the nervous system, the cardiovascular system, the immune system, the respiratory system and the metabolism, whereby these actions are in general of a suppressive nature, i.e., the actions are sedative, vasodilative, slow the heart frequency and inhibit the diuresis as well as the lipolysis.

To date, four different adenosine receptors have been identified that activate the adenylate cyclase via coupling to protein G and bear the names A₁, A_{2A}, A_{2B} and A₃. The central nervous system exhibits especially high densities of adenosine receptors, but in particular A₁- and A_{2B}-receptors are found in almost all tissue types. Because of their varied importance in medical physiology as well as pharmacology, adenosine receptors are in many cases subjects of detailed, scientific survey articles (see, e.g., K. N. Klotz, Naunyn Schmiedebergs Arch. Pharmacol., 362, 382-91 (2000); P. G. Baraldi et al., Med. Res. Rev., 20, 103-28 (2000); C. E. Müller, Farmaco, 56, 77-80 (2001), etc.). While receptor subtypes A₁ and A_{2A} are highly affine and are stimulated by adenosine already in nanomolar concentrations, subtypes A_{2B} and A₃ exhibit low (micromolar) affinity to natural ligands. On this basis, considerations of staggered activation of these receptors by increasing adenosine concentrations were advanced, whereby A₁-receptors provide the basal activation in physiological dormant concentrations, and A₃-receptors primarily exert their action for exceptional cases with greatly increased adenosine concentrations, for example for ischemic stroke or myocardial infarction.

Adenosine A₁-receptors are thus almost constantly activated according to this model presentation and exert a tonic inhibitory monitoring that can be eliminated by antagonists; this is, e.g., the pharmacological basis of the enlivening and dehydrating action of caffeine. Adenosine A₁-antagonists are therefore extremely advantageous as pharmacological active ingredients in many respects:
Within the scope of psychiatry for promoting the cognition for dementia conditions and as antidepressants;
In the cardiovascular and urological areas as antihypertensive agents and antiarrythmic agents;
For the therapy of acute renal failure, in which adenosine A₁-blocking eliminates the secondary vasoconstriction and can increase the blood supply. Here, the independent diuretic effect, especially the promoting of the potassium-saving natriuresis from A₁-antagonists is of additional importance, since water retention results in a further increased stress on the circulatory system.
In the lungs, adenosine A₁-antagonists can counteract a bronchoconstriction that is mediated via activation of A₁-receptors by relaxation of the smooth tracheal muscles; there are therefore potential anti-asthmatic agents. Since these active ingredients promote the ejection of chloride ions from the epithelial cells there, moreover, a positive action on the clinical picture of the mucoviscidosis is discussed.

In a remarkable manner, the activation of the A_{2A}-receptors in the brain as well as in the retina of the eye has an antagonistic effect on the A₁-receptors. A_{2A}-antagonists, which are brought into connection with the modulating action of the adenosine on the release of various neuropeptides, metabotropic and ionotropic glutamate, dopamine and nicotine receptors, which in turn again influence the release of acetylcholine and dopamine, as well as with the release of gamma-aminobutyric acid (GABA) (J. A. Ribeiro, Eur. J. Pharmacol., 375, 101-113 (1999)), and represent potential therapeutic agents for the treatment of Parkinson's disease, can therefore potentiate the action of cerebral A₁-agonists (F. Pedata et al., Ann. N.Y. Acad. Sci., 939, 74-84 (2001)). The latter represent potential active ingredients for the therapy of stroke patients and patients with retinal ischemia. Whether, however, adenosine A_{2A}-agonists can eliminate the action of adenosine A₁-inhibitors, however, is still not known at this time. Ligands with comparable bonding strength to Ax- and A_{2A}-receptors are considered undesirable, however, since the resulting pharmacological actions could be directed against one another.

It is known that adenosine receptor antagonists with a purine or xanthine partial structure exhibit neither adequate affinity nor selectivity. These parameters could, however, be significantly improved, for example by using 2-furanyl derivatives of the triazoloquinazoline, triazolopyrimidine and triazolotriazine with the following structural formulas (E. Ongini et al., Naunyn Schmiedebergs Arch. Pharmacol., 359, 7-10 (1999) and Farmaco 56, 87-90 (2001):

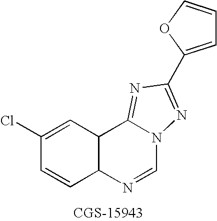

### 9-Chloro-2-(2-furanyl)-[1,2,4]triazolo[1,5-c]quinazoline-5-amine

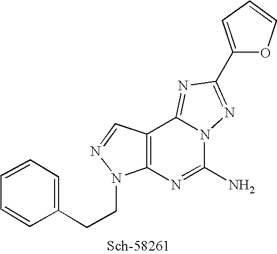

### 2-(2-Furanyl)-7-(2-phenylethyl)-7H-pyrazolo[4,3-e][1,2,4]triazolo[1,5-c]pyrimidine-5-amine

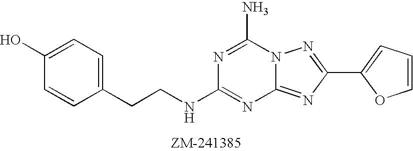

### 4-[2-[[7-Amino-2(2-furanyl)[1,2,4]triazolo[1,5-a][1,3,5]triazin-5-yl]amino]ethyl]-phenol

The cited examples, however, have different pharmacological or (physico)chemical drawbacks. The development of compound CGS-15943 as a therapeutic agent for ischemic stroke was thus halted because of inadequate selectivity. The compound Sch-52861, however, has a very high receptor affinity and good selectivity (K_{iA2A}=2.3 nm; K_{iA1}=121 nm), but the inadequate solubility and poor oral bioavailability limit the pharmaceutical suitability. The compound ZM-241385, which even has a selectivity factor of about 400, is used only as a tritated radioligand for visualization of A_{2A}-receptors in the animal test.

In addition to other triazolo[1,5-c]quinazolines and triazolo[1,5-c]pyrimidines, triazolo[4,3-a]quinoxalines were also examined. The fact that, on the one hand, 4-amino-6-benzylamino-1,2-dihydro-2-phenyl-1,2,4-triazolo[4,3-a]quinoxalin-1-one represents a selective A_{2A}-antagonist (V. Colotta et al., Arch. Pharm. Pharm. Med. Chem., 332, 39-41 (1999), and, on the other hand, 8-chloro-4-(cyclohexylamino)-1-(trifluoromethyl)[1,2,4]triazolo[4,3-a]quinoxaline (CP-68,247) exerts a highly selective antagonistic action on the A₁-receptor (IC₅₀ value is 28 nmol) (R. Sarges et al., J. Med. Chem., 33, 2240-2254 (1990)), clearly shows how closely these pharmacologically opposite actions lie to one another in this compound class. For all of these compounds, agonistic or partially agonistic action on the respective receptors is excluded, since such a one requires an intact nucleotide structure according to the present state of knowledge, but in any case an essentially intact ribose unit (C. E. Müller and B. Stein, Curr. Pharm. Design, 2,501-530 (1996) and literature cited therein).

Compounds with the following base are also known from the literature (B. Matuszczak et al., Arch. Pharm. Pharm. Med. Chem., 331, 163-169 (1998)).

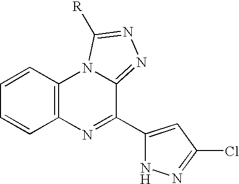

Also in this substance class, a dependence of the subtype-affinity on the substituent in 1-position was shown. While the 1-methyl derivative shows a higher affinity to the adenosine A_{2A}-receptor than to the A₁-receptor (A_{2A}: Ki=1.43 μm; A₁: Ki=7.85 μm, i.e., the selectivity factor is about 5), preference for the adenosine A₁-receptor is provided in the case of the other derivatives (for R=2-thienylmethyl: K_{iA1}=200 nm). A comparison of the absolute values of the binding affinity to that of other adenosine receptor antagonists shows, however, that the compounds of this type are inferior in this connection and therefore in all probability are not suitable as pharmaceutical agents.

### PRESENTATION OF THE INVENTION

The object of this invention is to further increase the receptor activities of the initially mentioned pyrazolyl-substituted triazoloquinoxalines as well as their analogs in the form of tetrazoloquinoxalines.

According to the invention, triazoloquinoxalines of general formula (I) with Y=CR, in particular 4-(chloropyrazolyl)-1-(3-phenylpropyl)-[1,2,4-]triazolo[4,3-a]quinoxaline as well as tetrazoloquinoxalines of general formula (2) with Y=N in 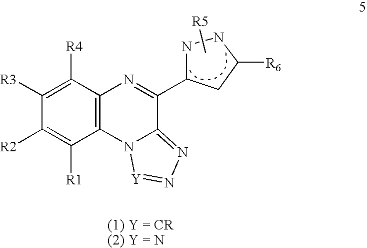
are proposed, in which R1 to R4 are hydrogen, linear or branched, saturated or unsaturated alkyl radicals, cycloalkyl radicals, which optionally have one or more heteroatoms, aryl or heteroaryl radicals, alkoxy, hydroxy, halogen, amino, nitro, trihalomethyl, carboxy, alkoxycarbonyl or sulfo groups, whereby R1 to R4 are identical or different or can be present as fused aryl or heteroaryl radicals or as correspondingly hydrogenated or partially hydrogenated systems, and in which substituent R is hydrogen or a linear or branched-chain, saturated, and/or unsaturated carbon radical, a cycloalkyl radical, an aryl or heteroaryl radical in substituted or unsubstituted form, whereby substituent R is bonded to the base either directly or via an alkylene group, in which one or more carbon atoms can be replaced by heteroatoms, such as oxygen, sulfur or nitrogen, and in which substituent R5 is hydrogen, C₁-C₈ alkyl, allyl, arylalkyl, (hetero)arylalkyl or acyl, and in which radical R6 is a halogen or hydrogen, excluding compounds with R1 to R5 equal to hydrogen, R6 equal to chlorine and R equal to methyl, phenyl, benzyl, 2-furyl, 2-thienyl or 2-thienylmethyl.

The first structure-activity relationships, which could be set up for this substance class, were in accordance with a pharmacophore model for A₁ antagonists that was described in the literature (B. Matuszczak et al., Arch. Pharm. Pharm. Med. Chem., 331, 163-169 (1998)). These results suggested that the structure-activity relationships of standard A₁-ligands are to be transferred to these tricyclic compounds. It should thus be possible, with the aid of known pharmacophore models, to develop compounds with improved receptor affinity as well as improved subtype selectivity.

It was shown, surprisingly enough, however, that the receptor affinities were no longer to reconcile other derivatives with the pharmacophore models. The finding that unchanged receptor affinity was provided after removal of individual structural components, which up until this time had been considered as essential to the design of the interactions of the ligand with the receptor, shows that an analogy with respect to the binding to the receptor is not given and thus structure-affinity relationships also cannot be transferred from other substance classes to these compounds. Pharmacophore models that are known in the literature thus also cannot be used in these pyrazolyl-substituted tricyclic compounds; this compound class is rather to be considered as a completely new invention.

Especially based on the pharmacophore and receptor model that is known in the literature, it was not to be expected that the compounds according to the invention show an affinity and selectivity to adenosine receptors, which makes them suitable to a large extent for use as pharmaceutical agents in terms of subtype-specific adenosine antagonists.

Analogs that are considered within the scope of the invention are, in the same way starting from general formulas (1) and (2), those compounds in which in each case independently of one another, it holds true that substituents R1 to R4 are hydrogen, linear or branched, saturated or unsaturated alkyl radicals, such as, for example, methyl, ethyl, propyl, butyl, isobutyl, allyl, or cycloalkyl radicals, whereby optionally one or more carbon atoms can be replaced by nitrogen, oxygen or sulfur, aryl or heteroaryl, alkoxy, hydroxy, halogen, amino, nitro, trihalomethyl, carboxy, alkoxycarbonyl or sulfo groups, whereby substituents R1 to R4 can be identical or different.

Substituent R can be hydrogen or an organic radical such as an alkyl, cycloalkyl, aryl or heteroaryl substituent, which is bonded to the base either directly or via an alkylene bridge, in which one or more carbon atoms can be replaced by heteroatoms, such as oxygen, sulfur or nitrogen.

As an organic radical, a linear or branched-chain C₁- to C₆-alkyl group, for example methyl, ethyl, propyl, butyl, or isobutyl, is preferred. This alkyl chain can also be substituted in turn, for example, with halogen, alkoxy, hydroxyl, amino—unsubstituted or substituted with one or two alkyl, aryl or acyl radicals—, alkoxycarbonyl, carboxyl, sulfo or cyano. In addition, this substituent can also contain one or more double or triple bonds, as is the case, for example, in allyl groups.

In addition, the organic radical can be a cycloalkyl substituent with the ring size that consists of three to eight carbon atoms. One or more carbon atoms in the ring can be replaced from heteroatoms, such as, for example, nitrogen, oxygen, or sulfur; moreover, the ring (for example, a piperazine, N-methylpiperazine, morpholine, dioxolane, dioxane, adamantane or noradamantane radical) can also be further substituted.

Further, an aryl or heteroaryl (for example furyl, thienyl, or pyridyl) radical is also preferred as an organic radical. The hetero(aryl) radical can optionally carry one or more substituents. As substituents of interest, in particular halogen, unsubstituted or else substituted alkyl—as an example of a substituted alkyl of interest, for example, trifluoromethyl, alkoxy, hydroxy, unsubstituted or substituted—for example also acylated—amino, alkxoycarbonyl, carboxyl, sulfo, nitro and cyano are used.

Substituent R5 can be hydrogen, a linear or branched alkyl, allyl, (hetero)arylalkyl, or acyl group. This substituent is found on one of the nitrogen atoms of the pyrazole ring.

Radical R6 can be either halogen, such as fluorine, chlorine, or bromine, or else hydrogen.

In addition, the invention relates to a process for the production of compounds (1) and (2), as well as pharmaceutical agents that contain the latter, as is disclosed according to the claims.

### METHODS FOR IMPLEMENTING THE INVENTION

In reference to the prior art, some possible variations are explained in the following experimental portion. These examples are used only for illustration, without, however, thus limiting the invention to this scope.

I. Process for the Production of Compounds According to Formula (1) While Varying Substituents R and R1 to R4

I.1 Symmetrically-Substituted Derivatives

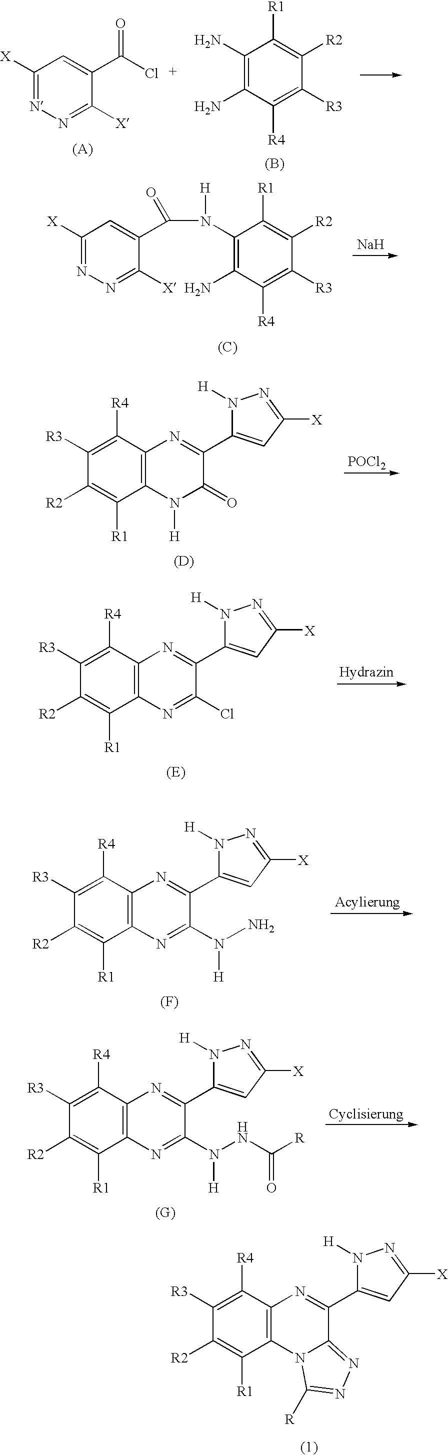

[Key:]
Hydrazin=Hydrazine
Acylierung=Acylation
Cyclisierung=Cyclization

X and X′ are the same radical or different radicals of the above-cited definition with the limitation X′=halogen, whereby in general formula (I), X also has the meaning of R6.

R, R1, R2, R3 and R4 can be defined according to the above-cited descriptions.

The synthesis sequence that is shown allows not only access to compounds in which the carbocyclic portion of the quinoxaline is unsubstituted, but also makes possible the production of symmetrically substituted compounds of the following type: 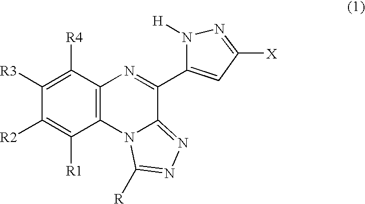
a) R1 to R4=identical substituents¹;
   ¹In this case, substituent means R≠H
b) R1 and R4=identical substituents and R2 and R3=H;
c) R2 and R3=identical substituents and R1 and R4=H;
d) R2 and R3=identical substituents and R1 and R4=identical substituents

The necessary symmetrically-substituted ortho-phenylenediamines can be purchased, are described in the literature or are synthetically available analogously to the derivatives that are described in the literature. Nitration reactions and subsequent reduction reactions are of special importance in the production of the substituted phenylenediamines.

The production of such symmetrically substituted compounds (with X=Cl and R1=R4=H and R2=R3=CH₃) is to be explained based on the dimethyl derivatives. The substance class that is selected for this purpose or the cited examples are used, however, only for illustration, without the invention being limited to their scope.

Below, synthesis examples are indicated for the production of the compounds that are shown in synthesis diagram 1:

### Synthesis of N-[2-Amino-4,5-dimethylphenyl)-3,6-dichloropyridazine-4-carboxamide* (C-1)

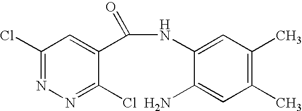 *Already described in: M. Banekovich, ‘Pyrazolyl-substituierte Chinoxaline: Darstellung neuer potentieller serotoninrezeptor-Liganden und Untersuchungen zum Fluoreszenz-Verhalten [Pyrazolyl-Substituted Quinoxalines: Production of New Potential Serotonin Receptor Ligands and Studies on Fluorescence Behavior],’ University Thesis, Innsbruck, 1998.

A solution that consists of 5.402 g (25.55 mmol) of 3,6-dichloropyridazine-4-carboxylic acid chloride in 60 ml of absolute dichloromethane is slowly added in drops to a suspension that consists of 10.440 g (76.65 mmol, 3 equivalents) of 4,5-dimethyl-o-phenylenediamine and 25.55 mmol of base (for example, triethylamine, pyridine, Hünig base, or the like) in 150 ml of absolute dichloromethane at 0° C. under nitrogen atmosphere. Then, the reaction batch is stirred until the reaction of the acid chloride is completed at room temperature (about 16 hours; TLC (thin-layer chromatogram) monitoring: several drops of the reaction batch are mixed with dilute hydrochloric acid, the solution is neutralized with saturated sodium bicarbonate solution and extracted with ethyl acetate, mobile solvent: ethyl acetate). The crystals that are produced are filtered off by suction, washed with dichloromethane and dried in a desiccator until a constant weight is reached.

The filtrate is extracted three times with 200 ml each of ice-cooled dilute sodium hydroxide solution, the aqueous phase is washed with dichloromethane and then acidified with concentrated hydrochloric acid while being cooled with ice. The resulting precipitate on diacylated product is filtered off by suction, the aqueous phase is washed twice with dichloromethane and then neutralized with saturated sodium bicarbonate solution. The neutral solution is exhaustively extracted with dichloromethane. The combined organic phases are washed with saturated sodium chloride solution, dried on sodium sulfate, filtered off and evaporated to the dry state.

For further reaction, the product (light yellow crystals) of the composition C₁₃H₁₂Cl₂N₄O (311.17) (yield: 90%) exhibits adequate purity.

| Melting point: | Starting from 198° C. decomposition | | | |
|---|---|---|---|---|
| Elementary analysis: | | C | H | N |
| | Cld. | 50.18% | 3.89% | 18.01% |
| | Fnd. | 50.22% | 4.12% | 17.96% |

| | |
|---|---|
| IR (KBr): | 3386, 3183, 3012, 1673cm⁻¹ |
| ¹H-NMR (DMSO-d₆) | 9.92(s, 1H, NH), 8.47(s, 1H, pyridazine H5), |
| | 7.01(s, 1H), 6.57(s, 1H)(H5, H6), 5.10(s, br, 2H, |
| | NH₂), 2.10(s, 3H, CH₃), 2.08(s, 3H, CH₃). |

### Synthesis of 3-[3(5)-Chloro-1H-pyrazol-5(3)-yl]-6,7-dimethylquinoxalin-2(1H)one* (D-1)

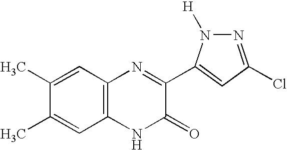

3 equivalents of base (preferably sodium hydride, 60% dispersion) is added to a solution that consists of 2.000 g (6.44 mmol) of N-(2-amino-4,5-dimethylphenyl)-3,6-dichloropyridazine-4-carboxamide (C-1) in 50 ml of anhydrous solvent (for example N,N-dimethylformamide) under nitrogen atmosphere, and the reaction mixture is stirred until the reaction is completed at 100° C. (the reaction time is about 15 minutes; for reaction monitoring, several drops of the reaction batch are mixed with dilute hydrochloric acid and extracted with ethyl acetate, mobile solvent: ethyl acetate; the complete reaction can be detected in addition in the color change of the reaction solution from dark red to brown).

For working-up, the reaction solution, while being exposed to nitrogen gassing, is carefully added to dilute hydrochloric acid. The accumulating product that is crystalline in this case is isolated and washed with water as well as petroleum ether and then dried until a constant weight is reached. The purification of the crude product is carried out by treatment of a solution of the substance in tetrahydrofuran with activated carbon in the heat, filtration and distillation of the solvent. The thus obtained product (yellow crystals) of the composition C₁₃H₁₁ClN₄O (274.71) (yield: 95%) has an adequate purity for the additional reaction.

| Melting point: | 328° C. while being decomposed | | | |
|---|---|---|---|---|
| Elementary analysis: | | C | H | N |
| | Cld. | 56.84% | 4.04% | 20.39% |
| | Fnd. | 56.75% | 4.34% | 20.12% |

| | |
|---|---|
| IR (KBr): | 3311, 1666cm⁻¹ |
| ¹H-NMR (DMSO-d₆) | 13.65(s, 1H, pyrazole NH), 12.60(s, |
| | 1H, quinoxaline NH), 7.51(s, 1H, quinoxaline |
| | CH,7.15(d, J=1.4Hz, 1H, pyrazole-CH), 7.07(s, |
| | 1H, quinoxaline CH), 2.29(s, 3H, CH₃), |
| | 2.27(s, 3H, CH₃). |

### Synthesis of 2-Chloro-3-[3(5)-chloro-1H-pyrazol-5(3)-yl]-6,7-dimethylquinoxaline* (E-1)

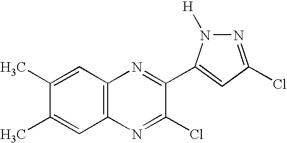

A suspension that consists of 2.601 g (9.47 mmol) of the corresponding quinoxalin-2-one derivative (D-1) is refluxed in a mixture that consists of 50 ml of phosphorus oxychloride and 5 ml of pyridine until the reaction is completed (about 3 hours; TLC monitoring: several drops of the reaction batch are mixed with saturated sodium bicarbonate solution and extracted with ethyl acetate, mobile solvent: ether). After cooling, the reaction mixture is carefully added to ice water. To improve the crystallization, for example, some saturated sodium bicarbonate solution is added. The precipitate is separated and washed with water as well as petroleum ether and dried in the desiccator until a constant weight is reached. For purification, a solution of the crude product in tetrahydrofuran is mixed with activated carbon, and this mixture is briefly heated to boiling, filtered off and evaporated to the dry state in a Rotavapor. The thus obtained product (beige-colored needles) of the composition C₁₃H₁₀Cl₂N₄ (293.16) exhibits an adequate purity for the additional reaction (yield: 1.868 g (67%)).

Already described in: M. Banekovich ‘Pyrazolyl-substituierte Chinoxaline: Darstellung neuer potentieller Serotoninrezeptor-Liganden und Untersuchungen zum Fluoreszenz-Verhalten,’ University Thesis, Innsbruck, 1998

| Melting point: | 296-300° C. | | | |
|---|---|---|---|---|
| Elementary analysis: | | C | H | N |
| | Cld. | 53.26% | 3.44% | 19.11% |
| | Fnd. | 53.39% | 3.61% | 19.23% |

| | |
|---|---|
| IR (KBr): | 3226cm⁻¹ |
| ¹H-NMR (DMSO-d₆) | 13.96(s, 1H, pyrazole NH), |
| | 7.89(s, 1H, quinoxaline CH, 7.84(s, |
| | 1H, quinoxaline CH), 7.19(s, 1H, pyrazole-CH), |
| | 2.50(s, 6H, 2xCH₃). |

Instead of a chloroquinoxaline (E), other derivatives can also be used for additional synthesis of compounds of type (F), in which the chlorine atom is replaced by another leaving group, for example by bromine, iodine or mesylate.

### Instead of 3-[3(5)-Chloro-1H-pyrazol-5(3)-yl]-6,7-dimethyl-2-hydrazinoquinoxaline (F-1)

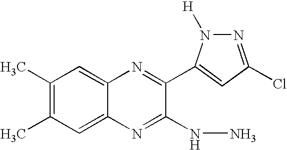

1.061 g (3.62 mmol) of the corresponding chloroquinoxaline derivative (E-1) is suspended in 20 ml of hydrazine monohydrate, and the mixture is refluxed until the reaction is completed (about 2 hours; TLC monitoring: several drops of the reaction batch are mixed with water and extracted with ethyl acetate, mobile solvent: ethyl acetate). After cooling, the reaction mixture is added to 150 ml of water, and to complete the crystallization, the resulting suspension is allowed to stand for several hours at about 4° C. The crystals are then isolated and washed with water as well as petroleum ether. After drying until a constant weight is reached, the crystals are dissolved in tetrahydrofuran, the solution is mixed with activated carbon and heated briefly to boiling, filtered, and evaporated to the dry state at reduced pressure. The product that is obtained (yellow powder) of composition C₁₃H₁₃ClN₆ (288.74) exhibits an adequate purity for the additional reaction; analytically pure compound is obtained by recrystallization from tetrahydrofuran.

| Yield: | 0.629 g(82%) | | | |
|---|---|---|---|---|
| Melting point: | 281-283° C. | | | |
| Elementary analysis: | | C | H | N |
| | Cld. | 54.08% | 4.54% | 29.11% |
| | Fnd. | 54.38% | 4.58% | 29.25% |

| | |
|---|---|
| IR (KBr): | 3320cm⁻¹ |
| ¹H-NMR (DMSO-d₆): | 7.54(s, 1H), 7.40(s, 1H)(H5, H8), |
| | 7.04(s, 1H, pyrazole H4), 2.36(s, 3H, |
| | CH₃), 2.33(s, 3H, CH₃) |

### General Operating Instructions for the Production of 2-Acylhydrazino-3-[3(5)-chloro-1H-pyrazol-5(3)-yl]-6,7-dimethylquinoxalines of type (G-1)

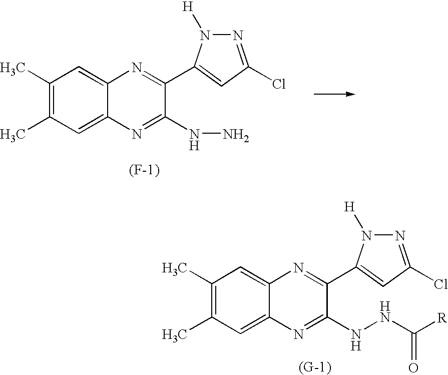

A solution of 1.1 equivalents of the corresponding acylating agent—for example acetyl chloride—in absolute 1,4-dioxane (about 2 ml per mmol of acylating agent) is slowly added in drops to a suspension that consists of one equivalent of hydrazinoquinoxaline (F-1) and 1.2 equivalents of base (for example, triethylamine, pyridine, and the like) in an absolute solvent (for example, 1,4-dioxane, THF, acetonitrile) (10 ml per mmol of hydrazino derivative F-1) at room temperature. After the addition is completed, the reaction batch is stirred at room temperature until the reaction is completed (about 12-18 hours; TLC monitoring: several drops of the reaction batch are mixed with water and extracted with ethyl acetate, mobile solvent: for example ethyl acetate). For working-up, the reaction solution is poured into water, and the resulting product is then isolated. A possibility of the isolation is the separation of the resulting precipitate and subsequent washing of the precipitate with water as well as petroleum ether. The isolation of the product by extraction with a suitable organic solvent represents an alternative; in this case, the solution of the acylated product is then washed with water and saturated sodium chloride solution as well as dried, and finally the organic solvent is removed by distillation.

To purify the respective acylated derivative, the corresponding product that is dried until a constant weight is reached and that consists of a suitable solvent is recrystallized. If necessary, the crude product is dissolved in advance in a suitable solvent—for example, tetrahydrofuran, mixed with activated carbon, boiled up, filtered off and evaporated to the dry state in a vacuum.

### Synthesis of 2-Acetylhydrazino-3-[3(5)-chloro-1H-pyrazol-5(3)-yl]-6,7-dimethylquinoxaline (G-1A)

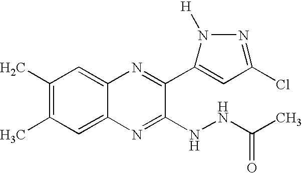

The production of the compound G-1A is carried out according to the general operating instructions for the production of compounds of type G-1 with use of the following reaction parameters or reactants:

| Acylating agent: | Acetyl chloride | | | |
|---|---|---|---|---|
| Reaction time: | 14 hours | | | |
| Appearance: | Yellow crystals | | | |
| Yield: | 61% | | | |
| Summation formula: | C₁₅H₁₅ClN₆O(330.78) | | | |
| Melting point: | 316-320° C.(consisting of ethyl | | | |
| | acetate-tetrahydrofuran(1:1)) | | | |
| Elementary analysis: | | C | H | N |
| | Cld. | 54.47% | 4.57% | 25.41% |
| | Fnd. | 54.39% | 4.59% | 25.15% |

| | |
|---|---|
| IR (KBr): | 3261, 1500cm⁻¹ |
| ¹H-NMR (DMSO-d₆ + D₂O): | 7.66(s, 1H), 7.47(s, 1H)(H5, H8), |
| | 7.09(s, 1H, pyrazole H4), 2.38(s, 3H, |
| | CH₃), 2.37(s, 3H, CH₃), |
| | 1.98(s, 3H, CH₃). |

### General Operating Instructions for Synthesis of 1-Substituted 4-[3(5)-Chloro-1H-pyrazol-5(3)-yl]-7,8-dimethyl-[1,2,4]triazolo [4,3-a]quinoxalines According to General Formula (1)

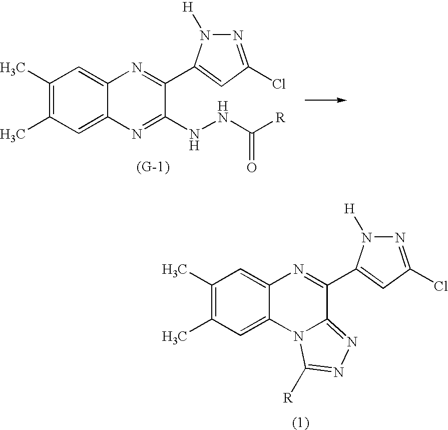

A suspension of acid hydrazide derivative (G-1) in a mixture that consists of 1,2-dichloroethane (20 ml per mmol) and phosphorus oxychloride (8 ml per mmol) is refluxed until the reaction is completed (about 2 hours; TLC monitoring: several drops of the reaction batch are mixed with water and extracted with ethyl acetate, mobile solvent: suitable organic solvent or organic mixture, for example ethyl acetate).

For working-up, the cooled reaction batch is carefully added to ice water, the resulting crystalline product is isolated and washed with water as well as petroleum ether. After drying, the product is dissolved in tetrahydrofuran, mixed with activated carbon, and the mixture is refluxed for several minutes. The filtrate is evaporated to the dry state in a vacuum, and the remaining substance is then recrystallized from a suitable solvent.

### Synthesis of 4-[3(5)-Chloro-1H-pyrazol-5(3)-yl]-1,7,8-trimethyl-[1,2,4]triazolo[4,3-a]quinoxaline (I.1.A.):

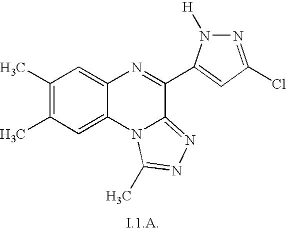

The synthesis of this compound is carried out by means of previously mentioned, general operating instructions, whereby the reaction time is two hours.

| Appearance: | Light yellow powder | | | |
|---|---|---|---|---|
| Yield: | 57% | | | |
| Summation formula: | C₁₅H₁₃ClN₆(312.76) | | | |
| Melting point: | 278-281° C.(consisting of ethyl acetate) | | | |
| Elementary analysis: | | C | H | N |
| (relative to | Cld. | 57.20% | 4.36% | 25.99% |
| C₁₅H₁₃ClN₆ x 0.1 | Fnd. | 57.00% | 4.27% | 25.98% |
| H₂O x 0.1 ethyl | | | | |
| acetate) | | | | |

| | |
|---|---|
| IR (KBr): | 3431cm⁻¹ |
| ¹H-NMR (DMSO-d₆): | 14.10(s, 1H, NH), 8.06(s, 1H), 7.80(s, |
| | 1H)(H6, H9), 7.54(s, 1H, pyrazole H4), 3.11(s, |
| | 3H, H₃), 2.45(s, 3H, CH₃), 2.39(s, 3H, |
| | CH₃). |

1.2 Unsymmetrically Substituted Derivatives

Since the reaction of unsymmetrically substituted ortho-phenylenediamines with 3,6-dihalo-pyridazine-4-carboxylic acid chloride results in general in the formation of an isomer mixture, and a separation of the isomer compounds is usually associated with a high expenditure of time and materials, the previously-described process does not allow any especially effective access to unsymmetrically substituted triazoloquinoxaline derivatives. The alternative synthesis strategy that is described below makes it possible, however, to obtain isomer-pure products and thus represents a considerable improvement compared to the current procedure.

This process according to the invention is distinguished in that primarily an N-acylation of an ortho-nitroaniline derivative is performed and only in the subsequent step is the second amino group formed. The latter is carried out by reduction of the nitro group. The additional reaction steps each correspond to the production of symmetrically substituted derivatives: 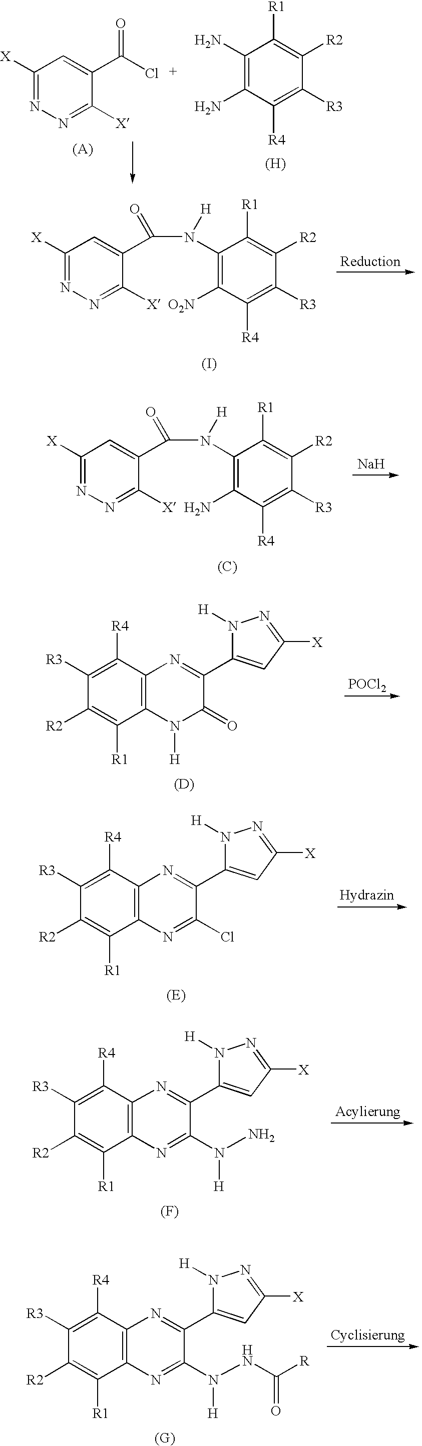 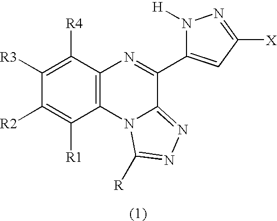
[Key:]
Reduktion=Reduction
Hydrazin=Hydrazine
Acylierung=Acylation
Cyclisierung=Cyclization

X and X′ are the same radical or a different radical of the above-cited definition with the limitation X′=halogen, whereby X has the meaning of R6.

R1, R2, R3, and R4 can be defined according to the above-cited descriptions.

The necessary substituted 2-nitroanilines can be purchased, are described in the literature or are synthetically available analogously to the derivatives that are described in the literature. Nitration reactions are of special importance here.

In the example of the monomethoxy derivative, this synthesis strategy is described in more detail below, but the invention is not to be limited to the latter.

Instead of ortho-nitroaniline derivatives, derivatives of substituted orthophenylenediamines can also be used, if one of the two nitrogen atoms carries a protective group, and thus it is ensured, on the one hand, that in the acylation only one isomer results, and, on the other hand, the protective group should be cleavable under extremely mild reaction conditions. Suitable protective groups are known from the literature; here, for example, N,O-acetals, N-benzyl, N-benzyloxycarbonyl, N-tert-butyl, N-[di-(p-methoxyphenyl)methyl]-, N-(2,4-dimethoxybenzyl)-, N-[9-phenylfluoren-9-yl]-, N-silyl derivatives or else imines can be used.

### Synthesis of N-(4-Methoxy-2-nitrophenyl)-3,6-dichloro-4-pyridazinecarboxamide (J-1)

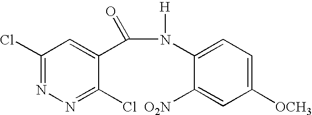

5.00 g (32.86 mmol) of 4-methoxy-2-nitroaniline is dissolved in 60 ml of absolute dichloromethane and mixed with 3.99 g (39.43 mmol, 1.2 equivalents) of triethylamine. After nitrogen is introduced, 7.30 g (34.51 mmol, 1.05 equivalents) of a solution of 3,6-dichloropyridazine-4-carboxylic acid chloride is slowly added in drops in several ml of absolute dichloromethane while being cooled with ice and while being stirred constantly. The reaction batch is then brought to room temperature, and stirring is continued until the reaction is completed (about 12 hours; TLC monitoring; mobile solvent: dichloromethane/ethyl acetate=9/1). For working-up, the reaction mixture is diluted with 50 ml of dichloromethane, and the organic phase is washed several times with 100 ml each of dilute hydrochloric acid. A portion of the product accumulates in crystalline form (TLC-pure) in this case and can be filtered off by suction.

The combined organic phases are-washed with water and saturated sodium chloride solution, dried on anhydrous sodium sulfate, filtered off and evaporated to the dry state in a Rotavapor. The residue is then recrystallized from ethyl acetate.

| | |
|---|---|
| Appearance: | Almost colorless powder |
| Yield: | 8.36 g(74%) |
| Melting point: | 198-201° C. |
| Summation formula: | C₁₂H₈Cl₂N₄O₄(343.13) |
| IR (KBr): | 1720cm⁻¹ |
| ¹H-NMR (DMSO-d₆): | 8.38(s, 1H, pyridazine H), 7.99(d, J=8.8Hz, |
| | 1H, H6), 7.74(d, J=2.9Hz, 1H, H3), 7.51(dd, |
| | J=8.8Hz, J=2.9Hz, 1H, H5), |
| | 3.88(s, 3H, OCH₃) |

### Reduction of N-(4-Methoxy-2-nitrophenyl)-3,6-dichloro-4-pyridazinecarboxamide (J-1)

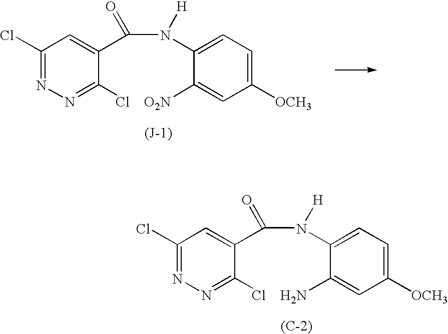

2.000 g (5.83 mmol) of N-(4-methoxy-2-nitrophenyl)-3,6-dichloro-4-pyridazinecarboxamide (J-1) is dissolved in 50 ml of tetrahydrofuran. 3.676 g (58.29 mmol, 10 equivalents) of ammonium formate and 0.864 g of palladium on activated carbon (10%) are added to this solution under nitrogen atmosphere, after which the reaction batch is stirred until the reaction is completed at room temperature (about 18 hours; TLC monitoring; mobile solvent: dichloromethane/ethyl acetate=30/1).

For working-up of the reaction batch, the catalyst is filtered off, and the filtrate is evaporated to the dry state. The residue is mixed with water, and the product is isolated by exhaustive extraction with ethyl acetate. The collected organic phases are washed with water as well as saturated sodium chloride solution and dried on sodium sulfate. After the solvent is distilled off, 1.403 g (77%) of a yellow powder of the composition C₁₂H₁₀Cl₂N₄O₂ remains, which can be further reacted without additional purification.

Melting point: 150-155° C.

¹H-NMR (DMSO-d₆): 9.86 (s, 1H, NH), 8.49 (s, 1H, pyridazine H5), 7.10 (d, J=8.8 Hz, 1H), 6.34 (d, J=2.6 Hz, 1H, H3), 6.19 (dd, J=8.8 Hz, J=2.6 Hz, 1H, H5), 5.08 (s, 2H, NH₂), 3.69 (s, 3H, OCH₃)

### Synthesis of 3-(3-Chloro-1H-pyrazol-5-yl)-6-methoxycuinoxalin-2(1H)-one (D-2)

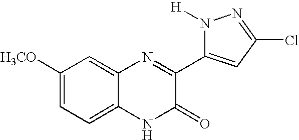

A solution of 0.695 g (2.22 mmol) of N-(2-amino-4-methoxyphenyl)-3,6-dichloro-4-pyridazine-carboxamide (C-2) in 25 ml of absolute N,N-dimethylformamide is mixed under nitrogen atmosphere with 0.266 g (6.66 mmol, 3 equivalents) of sodium hydride. The reaction mixture is stirred until the reaction is completed at 100° C. (reaction time: about 15 minutes; for reaction monitoring, several drops of the reaction batch are mixed with dilute hydrochloric acid and extracted with ethyl acetate, TLC monitoring of the extract mobile solvent: ethyl acetate; the complete reaction can be detected in addition from the changing of the reaction solution's color from dark red to brown).

For working-up, the reaction solution is carefully added under nitrogen atmosphere into 75 ml of dilute hydrochloric acid, after which the resulting suspension is allowed to stand for several hours at 4° C. The crystalline precipitating product is filtered off by suction and washed with water as well as petroleum ether. The residue that is dried until a constant weight is reached is dissolved for purification in tetrahydrofuran, the solution is mixed with activated carbon, and the mixture is briefly refluxed, filtered off and evaporated to the dry state in a Rotavapor. This product has adequate purity for the additional reaction. The analytically pure substance of the composition C₁₂H₉ClN₄O₂ (276.68) is obtained by recrystallization as a yellow powder from ethyl acetate.

| Yield: | 0.490 g(80%). | | | |
|---|---|---|---|---|
| Melting point: | 328-330° C. | | | |
| Elementary analysis: | | C | H | N |
| (relative to C₁₂H₉ClN₄O₂ x 0.1 | Cld. | 52.17% | 3.46% | 19.62% |
| ethyl acetate) | Fnd. | 52.47% | 3.62% | 19.49% |

| | |
|---|---|
| IR (KBr): | 3317, 2811, 1663(C=0)cm⁻¹ |
| ¹H-NMR (DMSO-d₆): | 13.74(s, 1H, pyrazole NH), 12.71(s, |
| | 1H, quinoxaline NH), 7.33-7.20(m, 4H, |
| | H5, H7, H8, pyrazole-H4), 3.83(s, 3H, |
| | OCH₃) |

### Synthesis of 2-Chloro-3-(3-chloro-1H-pyrazol-5-yl)-6-methoxyquinoxaline (E-2)

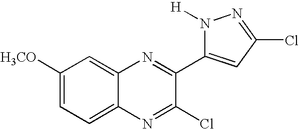

0.910 g (3.29 mmol) of the corresponding quinoxalin-2-one derivative (D-2) is dissolved in 15 ml of phosphorus oxychloride while being cooled with ice, and it is mixed with 1.5 ml of pyridine; the reaction mixture is then refluxed until the reaction is completed (about 3 hours; TLC monitoring: several drops of the reaction batch are mixed with saturated sodium bicarbonate solution and extracted with ethyl acetate, mobile solvent: ether). For working-up, the reaction batch is first cooled to 0° C., then carefully added to water and neutralized with saturated sodium bicarbonate solution to improve the crystallization. After several hours of standing at 4° C., the precipitate is separated. The analytically pure compound of composition C₁₂H₈Cl₂N₄O (295.13) is obtained by recrystallization from ethyl acetate in the form of yellow needles:

| Yield: | 0.660 g(68%). | | | |
|---|---|---|---|---|
| Melting point: | 243-246° C. | | | |
| Elementary analysis: | | C | H | N |
| | Cld. | 48.84% | 2.73% | 18.98% |
| | Fnd. | 49.04% | 2.98% | 18.76% |

| | |
|---|---|
| ¹H-NMR (DMSO-d₆): | 13.99(s, 1H, pyrazole NH), 7.98(d, J=9.0Hz, 1H, |
| | H8), 7.58(dd, J=9.0Hz, J=2.9Hz, 1H, H7), |
| | 7.45(d, J=2.9Hz, 1H, H5), 7.22(s, 1H, |
| | pyrazole-H4), 3.98(s, 3H, OCH₃) |

### Synthesis of 3-[3(5)-Chloro-1H-pyrazol-5(3)-yl]-2-hydrazino-6-methoxyquinoxaline (F-2)

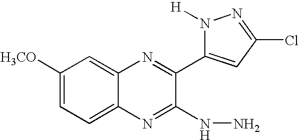

A suspension of 0.670 g (2.27 mmol) of 2-chloro-3-(3-chloro-1H-pyrazol-5-yl)quinoxaline (E-2) is suspended in 15 ml of hydrazine monohydrate, after which the reaction mixture is refluxed until the reaction is completed (reaction time: 2 hours, TLC monitoring: several drops of the reaction batch are mixed with water and extracted with ethyl acetate, mobile solvent: ether). For working-up, the reaction batch is added to 75 ml of water and allowed to stand for several hours at about 4° C. The precipitate that is produced is isolated, washed with water and petroleum ether and then dried in a vacuum until a constant weight is reached. For purification, the crude product is dissolved in tetrahydrofuran, mixed with activated carbon, and this mixture is refluxed for several minutes. After the activated carbon is removed, the solvent is distilled off, the resulting light residue is then suspended in ether, the solid is isolated and washed with ether. The thus obtained product of composition C₁₂H₁₁ClN₆O (290.71) exhibits adequate purity for the subsequent reaction. For analytical purposes, a recrystallization from ethyl acetate is carried out, which provides a beige-greenish powder in 60% yield (0.396 g).

| Melting point: | 219-222° C. | | | |
|---|---|---|---|---|
| Elementary analysis: | | C | H | N |
| (relative to C₁₂H₁₁ClN₆O x 0.2 | Cld. | 49.86 | 4.12 | 27.26 |
| ethyl acetate) | Fnd. | 50.16 | 4.16 | 27.01 |

| | |
|---|---|
| ¹H-NMR (DMSO-d₆ + D₂O): | 7.58(d, J=9.0Hz, 1H, H8), |
| | 7.28-7.25(m, 2H, H5, H7), 7.08(s, 1H, |
| | pyrazole-H4), 3.86(s, 3H, OCH₃) |

Introduction of an Acyl Radical into the Compound F-2 for the Production of Hydrazides of Type G-2

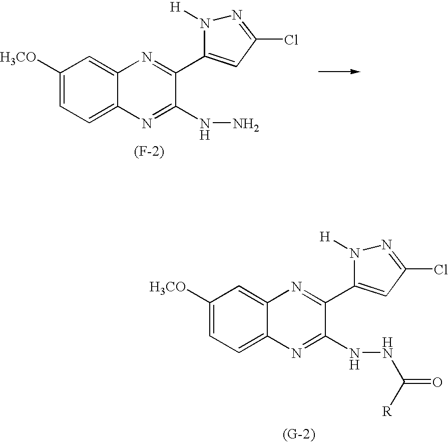

The synthesis instructions correspond to the general operating instructions for the production of compounds of type G-1.

The explanation by way of example is carried out on the following synthesis example:

### Synthesis of 3-[3(5)-Chloro-1H-pyrazol-5(3)-yl]-6-methoxy-2-(2-thienylacetyl)-hydrazinoquinoxaline (G-2A)

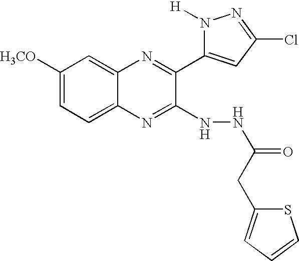

0.240 g (0.63 mmol) of hydrazinoquinoxaline F-2 is suspended in 30 ml of absolute 1,4-dioxane and mixed with 0.100 g (0.99 mmol, 1.2 equivalents) of triethylamine. Under protective gas atmosphere, a solution of 0.146 g (0.91 mmol, 1.1 equivalents) of 2-thienylacetyl chloride in 5 ml of absolute 1,4-dioxane is slowly added in drops at room temperature. The reaction solution is then stirred until the reaction is completed at room temperature (about 14 hours; for reaction monitoring, several drops of the reaction batch are mixed with dilute hydrochloric acid and extracted with ethyl acetate, mobile solvent: ethyl acetate). For working-up, the reaction batch is added to 75 ml of water, and the mixture is mixed with several milliliters of dilute hydrochloric acid to improve the crystallization.

After several hours of standing at about 4° C., the crystalline product is isolated and washed with water as well as petroleum ether. For purification, the dried substance is dissolved in tetrahydrofuran and mixed with activated carbon. This mixture is refluxed briefly, then it is filtered, and the solvent is removed by means of distillation. The product that is obtained in this way in 57% yield (yellowish-beige powder) of the composition C₁₈H₁₅ClN₆O₂S (414.88) exhibits an adequate purity for additional reaction.

### Synthesis of 1-Substituted 4-[3(5)-Chloro-1H-pyrazol-5(3)-yl]-7-methoxy-[1,2,4]triazolo [4,3-a]-quinoxalines of Type (1)

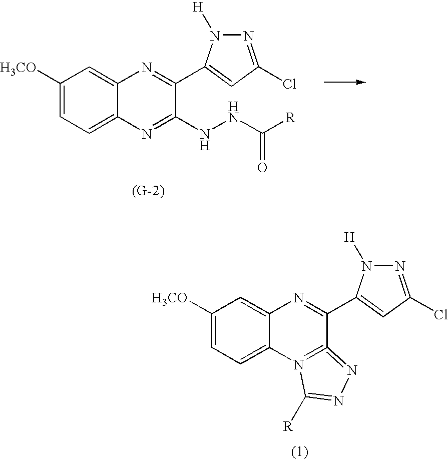

The synthesis corresponds to the operating instructions for cyclization of compounds of type G-1 and is explained in more detail in the following synthesis example.

### Synthesis of 4-[3(5)-Chloro-1H-pyrazol-5(3)-yl]-7-methoxy-1-(2-thienylmethyl)-[1,2,4]triazolo[4,3-a]-quinoxaline (I.2.A)

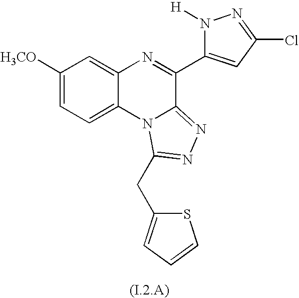

0.195 g (0.47 mmol) of acid hydrazide G-2A is refluxed in a mixture that consists of 5 ml of phosphorus oxychloride and about 15 ml of an inert solvent (e.g., 1,2-dichloroethane or acetonitrile) until the reaction is completed (about 2 hours; TLC monitoring: several drops of the reaction batch are mixed with water, the solution is neutralized with saturated sodium bicarbonate solution and extracted with ethyl acetate, mobile solvent: ethyl acetate).

After cooling, the reaction mixture is carefully added to 100 ml of ice water, the resulting crystals are isolated, washed with water and petroleum ether and dried. The aqueous filtrate is extracted three times with 100 ml each of ethyl acetate, the organic phase is washed with dilute sodium hydroxide solution, water, as well as saturated sodium chloride solution, and it is dried on sodium sulfate. The residue that remains after the solvent is distilled off is dissolved for purification in tetrahydrofuran, mixed with activated carbon and heated to boiling for a short time. The filtrate that is obtained after the activated carbon is removed is evaporated to the dry state. The crude product that is obtained is purified by means of column chromatography (stationary phase: silica gel; mobile phase: ethyl acetate) as well as recrystallization from ethyl acetate.

| Appearance: | Yellowish-brown powder | | | |
|---|---|---|---|---|
| Yield: | 0.170 g(91%) | | | |
| Summation formula: | C₁₈H₁₃ClN₆OS(396.86) | | | |
| Melting point: | 203-205° C. | | | |
| Elementary analysis: | | C | H | N |
| (Relative to C₁₈H₁₃ClN₆OS x | Cld. | 54.58% | 3.78% | 19.45% |
| 0.4 THF) | Fnd. | 54.45% | 3.70% | 19.39% |

| | |
|---|---|
| ¹H-NMR (CDCl₃): | 7.92(d, J=9.0Hz, 1H, H9), 7.90(s, 1H, |
| | pyrazole-4), 7.76(d, J=3.0Hz, 1H, H6), |
| | 7.23-7.20(m, 1H), 6.94-6.00(m, 1H), |
| | 6.79-6.78(m, 1H)(thiophene-H3, -H4, |
| | -H5), 7.13(dd, J=9.0Hz, J=3.0Hz, 1H, |
| | H8), 5.08(s, 2H, CH₂), 3.91(s, |
| | 3H, OCH₃) |

Depending on the substitution pattern, compounds can be further derivativized with substituents on the quinoxaline ring. These derivatizations comprise, for example, the processes that are known to one skilled in the art, such as acylation, alkylation, nitration, reduction, hydroxylation, ether cleavage, etc.

Unsymmetrically substituted derivatives can, if this substituent or substituents has/have influence on the acylation position, also be produced, moreover, starting from substituted ortho-phenylenediamines. The latter is provided, for example, when electron-withdrawing substituents are present. Starting from 4-nitrophenylenediamine, this variant of the production of unsymmetrically substituted compounds (for example with X, X′=Cl and R2=NO₂, R1=R3 R4=H) is to be illustrated below, without, however, thus limiting the invention to this scope. Additional reaction steps are performed as described above. 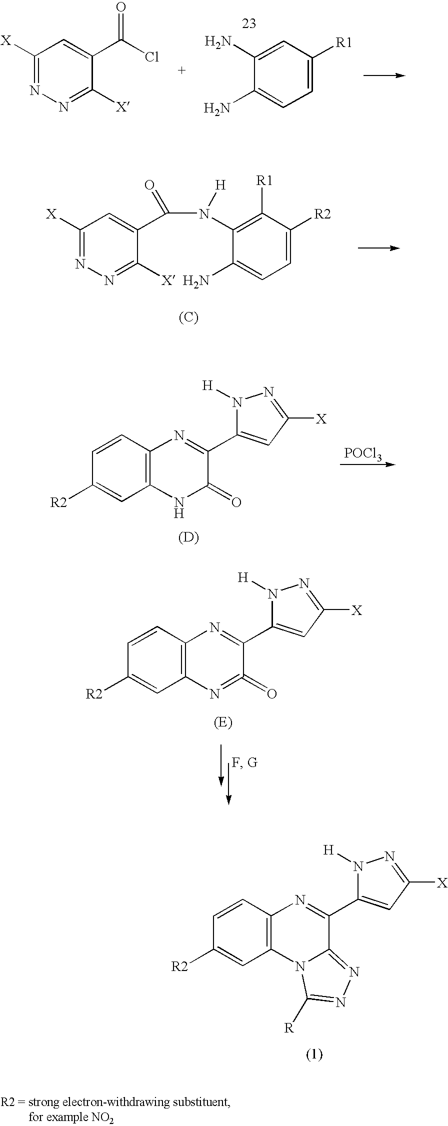
[R2=strong electron-withdrawing substituent, for example NO₂]

An analogous procedure can be used when substituents that have an opposite effect, i.e., activation of the amino group, are present. Both variants are also successful when using multiply-substituted phenylenediamines.

Depending on the substitution pattern, additional modifications can be performed later. Starting from the nitro group, in particular the reduction to the amino group is of special importance. This function can be further derivatized—e.g., acylated or alkylated; this function can also be modified via a diazonium salt.

The previously mentioned synthesis system is explained in more detail based on the following synthesis examples:

### Synthesis of N-(2-Amino-5-nitrophenyl)-3,6-dichoropyridazine-4-carboxamide (C-3)

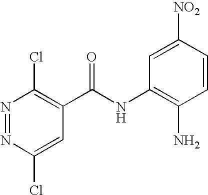

The solution of one equivalent of 3,6-dichloro-4-pyridazinecarboxylic acid chloride in absolute solvent is slowly added in drops to a mixture of equimolar amounts of 4-nitrophenylenediamine and base (for example, pyridine, triethylamine, Hünig base) in absolute solvent (e.g., tetrahydrofuran, dichloromethane, 1,4-dioxane, DMF, and the like) under nitrogen atmosphere and while being cooled with ice. The reaction batch is stirred at room temperature or elevated temperature, i.e., up to the boiling heat of the solvent that is used, until the reaction is completed. For acceleration, the reaction batch can also be treated for several minutes in ultrasound (TLC monitoring: several drops of the reaction batch are mixed with dilute hydrochloric acid, the solution is neutralized with saturated sodium bicarbonate solution and extracted with ethyl acetate, mobile solvent: suitable organic solvent, for example ethyl acetate). Then, the reaction product is isolated, which is carried out by separation of the crystals that are produced or by extraction with an organic solvent. Pure crystals are obtained by recrystallization.

| | |
|---|---|
| Appearance: | Yellowish-orange crystals |
| Melting point: | 244° C.(from 1,4-dioxane) |
| Yield: | 89% |
| Summation formula: | C₁₁H₇Cl₂N₅O₃(328.12) |
| IR (KBr): | 3448; 3259(NH₂); 1673(C═O) |
| ¹H-NMR (DMSO-d₆): | 10.41(s(br), 1H, NH, exchangeable with D₂O); |
| | 8.53(s, 1H, pyridazine-H5); 8.27(d, J=2.6Hz, |
| | 1H, phenyl-H3); 7.94(dd, J=9.2Hz; J=2.6Hz, |
| | 1H, phenyl-H5); 6.83(d, J=9.2Hz, 1H, phenyl- |
| | H6); 6.68(s, 2H, NH₂, exchangeable with D₂O) |

### Synthesis of 3-(5-Chloro-2H-pyrazol-3-yl)-7-nitro-1H-quinoxalin-2-one (D-3)

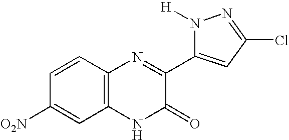

3 equivalents of base (preferably 60% sodium hydride dispersion) is added to a solution that consists of one equivalent of N-(2-amino-5-nitrophenyl)-3,6-dichloropyridazine-4-carboxamide (C-3) in an anhydrous solvent (for example, N,N-dimethylformamide) under nitrogen atmosphere, after which the reaction mixture is stirred until the reaction is completed at 100° C. (reaction time: about 15 minutes; for reaction monitoring, several drops of the reaction batch are mixed with dilute hydrochloric acid and extracted with ethyl acetate; mobile solvent: ethyl acetate; the completed reaction can be detected in addition from the changing of the reaction solution's color from dark red to brown).

| | |
|---|---|
| Appearance: | Yellow crystals |
| Melting point: | 340° C. |
| Yield: | 84% |
| Summation formula: | C₁₁H₆ClN₅O₃(291.66) |
| IR(KBr): | 3274(NH₂); 1687(C═O) |
| ¹H-NMR(DMSO-d₆): | 13.96(s, 1H, NH); 13.02(s, 1H, NH); |
| | 8.11-7.93(m, 3H, phenyl-H); 7.28(s, |
| | 1H, pyrazole-CH) |

The additional reaction to the compound of general formula (I) is carried out with the formation of the compounds of general formulas (E), (F) and (G) as intermediate products, which can also be produced just like the already described compounds (E-1), (F-1) and (G-1).

II. Process for the Production of Compounds of Type (1) While Varying Substituent R:

II.1 Derivatives with Different Substituents in 1-Position

Synthesis strategy that is described above and that can be performed analogously to the prior art can be used for the production of the compounds according to the invention with different substituents in 1-position (for R≠H). Depending on the substitution pattern, further derivatizations, such as the processes of oxidation, reduction, ether cleavage, acylation, alkylation, hydrolysis, etc., that are known to one skilled in the art can be performed. The production of the compounds according to the invention with different substituents in 1-position is explained based on the examples below, but it is not limited to these examples. Representatives that carry an alkyl, cycloalkyl, aryl or heteroaryl substituent are presented here, whereby this radical is connected directly or via a spacer to the tricyclic compound.

II.1.1 Examples of Derivatives with (Substituted) Alkyl Radicals in 1-Position

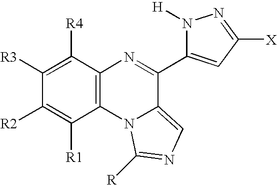

| | | | | | | Recrystallization |
|---|---|---|---|---|---|---|
| | | | | Summation | Medium | |
| | | | | Formula | Appearance | |
| No. | X | R1-R4 | R | (MG) | Melting Point | ¹H-NMR |
| II.1.1A | Cl | H | —CH₂CH₃ | C₁₄H₁₁ClN₆ × | Ethyl Acetate | 14.19(s, 1H, Pyrazole |
| | | | | 0.5 H₂O | Light Pink Crystals | NH), 8.33-8.29(m, 1H), |
| | | | | (307.74) | 247-250° C. | 8.11-8.07(m, 1H), 7.82- |
| | | | | | | 7.69(m, 2H) |
| | | | | | | (H6/H7/H8/H9), 7.57(s, |
| | | | | | | 1H, pyrazole H4), 3.51(q; |
| | | | | | | J=7.4 Hz,, 2H, CH₂), 1.51 |
| | | | | | | (t, J=7.4 Hz, 3H, CH₃) |
| II.1.1B | Cl | H | —CH₂CH₂— | C₁₅H₁₃ClN₆ × | Ethyl Acetate | 14.20(s, 1H Pyrazole- |
| | | | CH₃ | 0.6 HCl | Colorless Crystals | NH), 8.34-8.30(m, 1H), |
| | | | | (334.64) | 230-233° C. | 8.14-8.10(m, 1H), 7.85- |
| | | | | | | 7.70(m, 2H) |
| | | | | | | (H6/H7/H8/H9), 7.60(s, |
| | | | | | | 1H, pyrazole-H4), 3.48(t; |
| | | | | | | J=7.2 Hz, 2H, CH₂), |
| | | | | | | 2.05-1.94(m, 2H, |
| | | | | | | CH₂), 1.11(t, |
| | | | | | | J=7.3 Hz, 3H, CH₃) |
| II.1.1C | Cl | H | —(CH₂)₃CH₃ | C₁₆H₁₅ClN₆ × | Ethyl Acetate | 14.19(s, 1H, Pyrazole- |
| | | | | 0.2 H₂O | Light Pink Crystals | NH), 8.33-8.29(m, 1H), |
| | | | | (330.39) | 258-260° C. | 8.12-8.08(m, 1H), 7.84- |
| | | | | | | 7.59(m, 2H) |
| | | | | | | (H6/H7/H8/H9), 7.59(s, |
| | | | | | | 1H, pyrazole-H4), 3.49(t, |
| | | | | | | J=7.4 Hz, 2H, |
| | | | | | | CH₂), 2.02- |
| | | | | | | 1.87(m, 2H, CH₂), 1.63- |
| | | | | | | 1.44(m, 2H, CH₂), 0.98(t, |
| | | | | | | J=7.2 Hz, 3H, CH₃) |
| II.1.1D | Cl | H | —CH₂CH— | C₁₆H₁₅ClN₆ | Ethyl Acetate | 14.19(s, 1H, Pyrazole- |
| | | | (CH₃)₂ | (326.79) | Colorless to | NH), 8.29-8.26(m, 1H), |
| | | | | | Yellowish Needles | 8.12-8.07(m, 1H), 7.85- |
| | | | | | 211-215° C. | 7.69(m, 2H) |
| | | | | | | (H6/H7/H8/H9), 7.59(s, |
| | | | | | | 1H, pyrazole-H4), 3.38(d, |
| | | | | | | J=7.0 Hz, 2H, CH₂), 2.44- |
| | | | | | | 2.31(m, 1H, CH), 1.08(d, |
| | | | | | | J=6.4 Hz, 6H, 2 × CH₃) |
| II.1.1E | Cl | H | 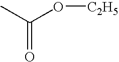 | C₁₅H₁₁ClN₆O₂ (342.75) | Tetrahydrofuran Light Yellow Crystals 327-331° C. | 7.96-7.92(m, 2H), 7.79-7.72(m, 1H), 7.65-7.57 (mm 1H) (H6/H7/H8/H9), 6.89(s, 1H, pyrazole-H4), 4.26(q, J=6.9 Hz, 2H, CH₂), 1.24(t, J=6.9 Hz, 3H, CH₃) |
| II.1.1F | Cl | H | 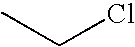 | C₁₃H₈Cl₂N₆(319.15) | Colorless to Yellowish Needles 294-255° C. | 14.30(s, 1H, Pyrazole- NH), 8.44-8.40(m, 1H), 8.19-8.15(m, 1H), 7.90-7.81(m, 2H) (H16/H7/H8/H9), 7.62(s, 1H, pyrazole H4), 5.74(s, 2H, CH₂) |

II.1.2 Examples of Derivatives with Cycloalkyl Substituents in 1-Position

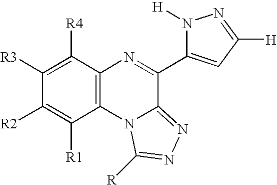

| | | | | | Recrystallization | |
|---|---|---|---|---|---|---|
| | | | | Summation | Medium | |
| | | | | Formula | Appearance | |
| No. | X | R1-R4 | R | (MG) | Melting Point | ¹H-NMR |
| II.1.2A | Cl | H | 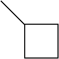 | C₁₃H₁₃ClN₆ ×0.7 HCl (350.30) | Ethyl Acetate Light Pink Crystals 238-240° C. | 14.21(s, 1H, Pyrazole- NH), 8.17-8.08(m, 2H), 7.85-7.69(m, 2H) (H6/H7/H8/H9), 7.61(s, 1H, pyrazole-H4), 4.49-4.34(m, 1H, CH), 2.70-2.59(m, 4H, 2 × CH₂), 2.29-1.98(m, 2H, CH₂) |
| II.1.2B | Cl | H | 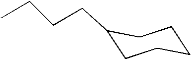 | C₂₁H₂₃ClN₆(394.91) | Colorless to Yellowish Needles 222-223° C. | 14.23(s, 1H, Pyrazole- NH), 8.32-8.29(m, 1H), 8.13-8.09(m, 1H), 7.84-7.70(m, 2H) (H6/H7/H8/H9), 7.60(s, 1H, pyrazole-H-4), 3.47(t, J=7.3 Hz, 2H, CH₂), 2.05-1.87(m, 2H, CH₂), 1.74-1.63(m, 5H, CH, 2 × CH₂), 1.47-1.13(m, |
| # 6H, 3 × CH₂), 0.96-0.86 (m, 2H, CH₂) | | | | | | |
| II.1.2C | Cl | H | 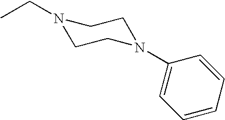 | C₂₃H₂₁Cl₂N₈(444.93) | Colorless to Yellowish Needles 235-240° C. | 14.25(s, 1H, Pyrazole- NH), 8.53-8.49(m, 1H), 8.14-8.10(m, 1H), 7.79-7.71(m, 2H) (H6/H7/H8/H9), 7.61(s, 1H, pyrazole-H-4), 7.27 (s br., 5H, phenyl-H), 4.28(s, 2H, CH₂), 3.42(s, 2H, CH₂), 2.58(“s” br., 4H, piperazine-CH), 2.35(‘s’br., |
| # br., 4H, piperazine-CH) | | | | | | |
| II.1.2D | Cl | H | 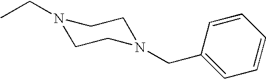 | C₂₄H₂₁Cl₂N₈(458.96) | Colorless to Yellowish Needles 245-258° C. | 14.3(s, 1H, Pyrazole-NH), 8.53-8.49(m, 1H), 8.14-8.10(m, 1H), 7.79-7.71(m, 2H)(H6/H7/H8/H9), 7.61 (s, 1H, pyrazole-H-4), 7.3 (br., 5H, phenyl-H), 5.2(s, 2H, CH₂), 4.3(s, 2H, CH₂), 3.42(s, 2H, CH₂), 2.6(‘s’br., 4H, |
| # piperazine-CH), 2.4(‘s’br., 4H, piperazine-CH) | | | | | | |

II.1.3 A) Examples of Derivatives with (Substituted) Aryl Radicals in Position

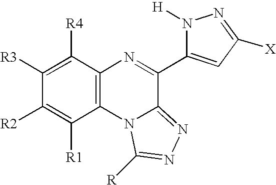

| | | | | | Recrystallization | |
|---|---|---|---|---|---|---|
| | | | | Summation | Medium | |
| | | | | Formula | Appearance | |
| No. | X | R1-R4 | R | (MG) | Melting Point | ¹H-NMR |
| II.1.3A | Cl | H | 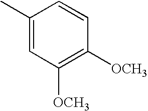 | C₂₀H₁₅ClN₆O₂(406.83) | Ethyl Acetate Light Yellow Crystals 257-267° C. | 14.29(s, 1H, Pyrazole- NH), 8.13(d, J=8.6 Hz, 1H), 7.71-7.58(m, 4H), 7.35-7.22(m, 3H) (H6/H7/H8/H9, pyrazole- H-4, phenyl-H), 3.91(s, 3H, OCH₃), 3.76(s, 3H, OCH₃) |
| II.1.3B | Cl | H | 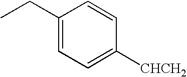 | C₂₀H₁₅ClN₆O (390.83) | Ethyl Acetate Colorless Crystals 240-247° C. | 14.25(s, 1H, Pyrazole- NH), 8.20-8.07(m, 2H), 7.73-7.60(m, 2H) (H6/H7/H8/H9), 7.64(s, 1H, pyrazole-H-4), 717(d, J=8.7 Hz, 2H, phenyl-H), 6.86(d, 8.7 Hz, 2H, phenyl-H), 4.92(s, 2H, CH₂), 3.69 (s, 3H, CH₃) |
| II.1.3C | Cl | H | 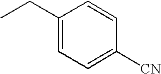 | C₁₉H₁₃ClN₆O ×0.9 H₂O (393.02) | Ethyl Acetate/THF Colorless Crystals 306-314° C. | 14.24(s, 1H, Pyrazole- NH), 9.31(s, 1H, OH), 8.50-8.05(m, 2H), 7.75-7.55(m, 3H) (H6/H7/H8/H9, pyrazole- H-4), 7.03(d, J=6.1 Hz, 2H, phenyl-H), 6.68 (d, J=6.1 Hz, 2H, phenyl-H), 4.86(s, 2H, CH₂) |
| II.1.3D | Cl | H | 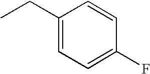 | C₁₉H₁₂ClFN₆(378.80) | Colorless to Yellowish Needles 257-260° C. | 14.27(s, 1H, Pyrazole- NH), 8.20-8.08(m, 2H), 7.71-7.67(m, 2H) (H6/H7/H8/H9), 7.63(s, 1H, pyrazole-H-4), 7.35-7.28(m, 2H, phenyl-H), 7.28-7.10(m, 2H, phenyl- H), 4.99(s, 2H, CH₂) |
| II.1.3E | Cl | H | 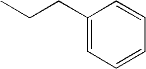 | C₂₀H₁₅ClN₆(374.83) | Ethyl Acetate/THF Slightly Grayish Crystals 264-272° C. | 14.28 (s, 1H, Pyrazole- NH), 8.38-8.34(m, 1H) 8.15-8.10(m, 1H), 7.84-7.67(m, 2H) (H6/H7/H8/H9), 7.61(s, 1H, pyrazole-H-4), 7.42-7.19(m, 5H, phenyl-H), 3.91-3.76(m, 4H, 2 × CH₂) |
| II.1.3F | Cl | H | 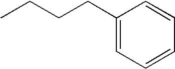 | C₂₁H₁₇ClN₆(388.86) | Ethyl Acetate Light Beige Crystals 203-208° C. | 14.20(s, 1H, Pyrazole- NH), 8.13-8.10(m, 2H), 7.78-7.71(m, 2H) (H6/H7/H8/H9), 7.60(s, 1H, pyrazole-H-4), 7.28-7.23(m, 5H, phenyl-H), 3.51(t, J=7.2 Hz, 2H, CH₂), 2.85(t, J=7.3 Hz, 2H, CH₂), 2.32-2.18(m, 2H, CH₂) |
| II.1.3G | Cl | H | 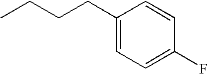 | C₂₁H₁₆ClFN₆(406.85) | Colorless to Yellowish Needles 228-230° C. | 14.19(s, 1H, Pyrazole- NH), 8.20-8.07(m, 2H), 7.75-7.71(m, 2H) (H6/H7/H8/H9), 7.58(s, 1H, pyrazole-H-4), 7.35-7.27(m, 2H, phenyl-H), 7.15-7.06(m, 2H, phenyl- H), 3.49(t, J=7.3 Hz, 2H, CH₂), 2.83(t, J=7.5 Hz; 2H, CH₂), 2.30-2.19 (m, 2H, CH₂) |
| II.1.3H | Cl | H | 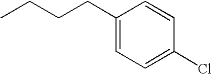 | C₂₁H₁₆Cl₂N₆(423.30) | Colorless to Yellowish Needles 200-210° C. | 14.21(s, 1H, Pyrazole- NH), 8.25-8.10(m, 2H), 7.78-7.74(m, 2H) (H6/H7/H8/H9), 7.62(s, 1H, pyrazole-H-4), 7.38-7.28(m, 5H, phenyl-H), 3.53(t, J=7.1 Hz, 2H, CH₂), 2.85(t, J=7.7 Hz, 2H, CH₂), 2.32-2.18(m, 2H, CH₂) |
| II.1.3I | Cl | H | 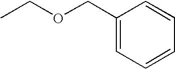 | C₂₀H₁₅ClN₆O (390.83) | Ethyl Acetate Colorless Crystals 217-227° C. | 14.26(s, 1H, Pyrazole- NH), 8.39-8.35(m, 1H), 8.16-8.11(m, 1H), 7.85-7.75(m, 2H) (H6/H7/H8/H9), 7.63(s, 1H, pyrazole-H-4), 7.34-7.25(m, 5H, phenyl-H), 5.36(s, 2H, CH₂), 4.68(s, 2H, CH₂) |
| II.1.3J | Cl | H | 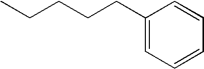 | C₂₂H₁₉ClN₆(402.89) | THF Colorless to Yellowish Needles 210-215° C. | 14.23(s, 1H, Pyrazole- NH), 8.31-8.29(m, 1H), 8.13-7.83(m, 1H), 7.79-7.66(m, 2H) (H6/H7/H8/H9), 7.60(s, 1H, pyrazole-H-4), 7.30-7.13(m, 5H, phenyl-H), 3.52(t, J=7.0 Hz, 2H, CH₂), 2.60(t, J=7.3 Hz, 2H, CH₂), 2.02-1.78(m, 4H, 2 × CH₂) |
| II.1.3K | Cl | H | 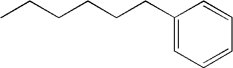 | C₂₃H₂₁ClN₆(458.96) | THF Colorless to Yellowish Needles 210° C. | 14.22(s, 1H, Pyrazole- NH), 8.33-8.28(m, 1H), 8.13-8.08(m, 1H), 7.84-7.69(m, 2H) (H6/H7/H8/H9), 7.59(s, 1H, pyrazole-H-4), 7.28-7.13(m, 5H, phenyl-H), 3.48(t, J=7.4 Hz, 2H, CH₂), 2.60(t, J=7.3 Hz, 2H, CH₂), 2.07-1.91(m, 2H, CH₂), |
| # 1.76-1.45(m, 4H, 2 × CH₂) | | | | | | |
| II.1.3L | Cl | H | 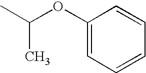 | C₂₀H₁₅ClN₆O ×0.4 H₂O (398.04) | Ethyl Acetate Light Yellow Crystals 225-230° C. | 14.26(s, 1H, Pyrazole- NH), 8.40-8.30(m, 1H), 8.20-8.12(m, 1H), 7.78-7.70(m, 2H) (H6/H7/H8/H9), 7.63(s, 1H, pyrazole-H-4), 7.36-7.29(m, 2H), 7.14-6.96(m, 2H)(phenyl-H), 6.62(d, J=5.8 Hz, 1H), 4.06-3.96 (m, 1H, α-CH), |
| # 1.96(d, J=7.6 Hz, 3H, CH₃) | | | | | | |

II.1.3 B) Examples of Derivatives with Heteroaryl Substituents in 1-Position

| | | | | | Recrystallization | |
|---|---|---|---|---|---|---|
| | | | | Summation | Medium | |
| | | | | Formula | Appearance | |
| No. | X | R1-R4 | R | (MG) | Melting Point | ¹H-NMR |
| II.1.3M | Cl | H | 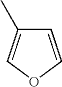 | C₁₆H₉ClN₆O (336.74) | Ethyl Acetate Beige-Gray Crystals 306-309° C. | 14.28(s, 1H, Pyrazole- NH), 8.37(“t,”1H), 8.16-8.09(m, 2H), 7.91-7.86(m, 1H), 7.76-7.62(m, 3H), 7.01-7.00(m, 1H)(H-6/-7/ -8/-9, pyrazole-H4, furyl- H) |
| II.1.3N | Cl | H | 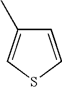 | C₁₆H₉ClN₆S ×0.1 THF × 0.9 H₂O (376.23) | Ethyl Acetate/THF Light Yellow Crystals 297-300° C. | 14.2(br s, 1H, Pyrazole- NH), 8.22-8.21(m, 1H), 8.14-8.10(m, 1H), 7.98-7.96(m, 1H), 7.69-7.45(m, 5H)(H6/H7/H8/H9, pyrazole-H4, thienyl- H2/H4/H5) |
| II.1.3O | Cl | H | 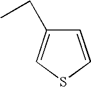 | C₁₇H₁₁ClN₆S ×0.6 H₂O (377.64) | Ethyl Acetate Beige Crystals 240-245° C. | 14.24(s, 1H)(Pyrazole- NH), 7.25(s, 1H, pyrazole- H4), 8.20-8.08(m, 2H), 7.74-7.48(m, 3H), 7.09-7.07(m, 1H), (H6/H7/H8/H9, thienyl- H2/H4/H5), 4.96(s, 2H, CH₂) |
| II.1.3P | Cl | H | 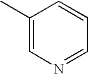 | C₁₇H₁₀ClN₇(347.77) | Ethyl Acetate/THF Yellow Crystals Mod 1. 314-318° C. Mod. 2 > 350° C. | 14.33(s, 1H, Pyrazole- NH), 9.00-8.95(m, 1H), 8.69-8.14(m, 3H), 8.00-7.90(m, 1H), 7.80-7.38(m, 4H)(H6/H7/H8/H9, pyrazole-H-4, pyridine-H) |
| II.1.3Q | Cl | H | 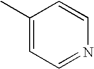 | C₁₇H₁₀ClN₇(347.77) | Ethyl Acetate Orange Crystals Mod. 1 270-278° C. Mod. 2 > 350° C. | 8.56(s, 2H), 7.92-7.50(m, 6H)(H6/H7/H8/H9, Pyridine-H), 6.84(s, 1H, pyrazole-H-4) |

II.2 Derivatives of the Compounds According to General Formula (I) with Hydrogen Atoms in 1-Position:

Derivatives in which substituent R (1-position) represents a hydrogen atom cannot be obtained according to the previously-described methods. The latter are reacted, for example, by reaction of the corresponding hydrazino compound with trimethyl orthoformate. Below, general operating instructions, which explain the formation of such derivatives, as well as three examples, are cited. 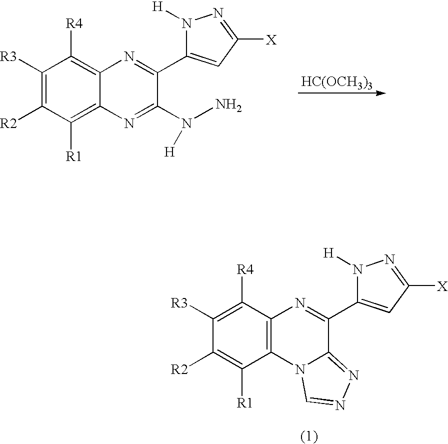
General Operating Instructions:

A suspension that consists of 2.0 mmol of the corresponding hydrazino compound in trimethyl orthoformate (about 10 ml per mmol) is refluxed until the reaction is completed (TLC monitoring: several drops of the reaction batch are mixed with water, the product is extracted with ethyl acetate, mobile solvent: ethyl acetate).

After cooling to room temperature, the reaction product is isolated. This is carried out either by the crystallized product being filtered off by suction or by mixing the reaction batch with water and extraction with a suitable organic solvent (for example, dichloromethane). The isolated crude product is then purified by recrystallization from a suitable solvent, for example ethyl acetate, tetrahydrofuran, 1,4-dioxane, N,N-dimethylformamide or alcohols.

### II.2.A. Synthesis of 4-[3(5)-Chloro-1H-pyrazol-5(−3)-yl-[1,2,4]triazolo[4,3-a]-quinoxaline (a Compound of General Formula (1) with R=R1===R3=R4=H, X=

| | |
|---|---|
| Reaction time: | 2 hours |
| Yield: | 80%(colorless needles) |
| Summation formula: | C₁₂H₇ClN₆(270.68) |
| Melting point: | 302-304° C.(from THF + 5% DMF) |
| ¹H-NMR (DMSO-d₆): | 14.24(s, 1H, pyrazole NH), 10.21(s, |
| | 1H, H1), 8.49-8.44(m, 1H), 8.14-8.08(m, |
| | 1H), 7.88-7.70(m, 2H)(H6, H7, H8, H9) |

### II.2.B. Synthesis of 4-[3(5)-Chloro-1H-pyrazol-5(3)-yl]-7,8-dimethyl-[1,2,4]triazolo[4,3-a]-quinoxaline (A Compound of General Formula (1) with R=R1=R4=H, R2=R3=CH₃, X=Cl)

| | |
|---|---|
| Reaction Time: | 40 minutes |
| Yield: | 90%(colorless powder) |
| Summation formula: | C₁₄H₁₁ClN₆(298.74) |
| Melting point: | 320-323° C.(from THF) |
| ¹H-NMR(DMSO-d₆): | 14.13(s, 1H, pyrazole NH), 10.04(s, |
| | 1H<H1), 8.17(s, 1H), 7.75(s, 1H)(H6, H9), |
| | 7.56(s, 1H, pyrazole-H4), 2.41(s, 3H, |
| | CH₃), 2.38(s, 3H, CH₃) |

### II.2.C. Synthesis of 4-[3(5)-Chloro-1H-pyrazol-5(3)-yl]-7-methoxy-[1,2,4]triazolo[4,3-a]-quinoxaline (A Compound of General Formula (1) with R=R1, R2, R4=H, R═OCH₃, X=Cl)

| | |
|---|---|
| Reaction time: | 4 hours |
| Yield: | 67%(yellowish powder) |
| Summation formula: | C₁₃H₉ClN₆O(300.71) |
| Melting point: | 287-290° C.(starting from 200° C. |
| | conversion into another modification)(from |
| | ethyl acetate/THF) |
| ¹H-NMR (DMSO-d₆): | 14.21(s, 1H, pyrazole NH), 10.14(s, |
| | 1H, H1), 8.40(d, J=9.0Hz, 1H, H9), |
| | 7.64(s, 1H, pyrazole-H4), 7.55(d, J=2.9Hz, |
| | 1H, H6), 7.47(dd, J=9.0Hz, J=2.9Hz, 1H, H8), |

3.94 (s, 3H, OCH₃)
   II.3 Tetrazoloquinoxalines of General Formula (2). (1-Aza Derivatives):

In addition, the invention relates to new tetrazoloquinoxalines that can be considered as N-isoteres of the previously cited triazoloquinoxalines according to general formula (I), i.e., a ring-carbon was replaced by nitrogen. Such derivatives are, as indicated based on an example, accessible by reaction with sodium azide starting from the corresponding chloroquinoxaline derivatives. 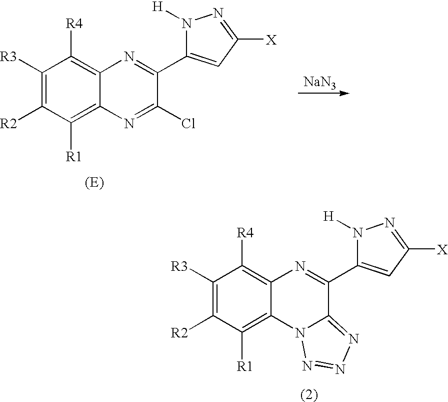
General Operating Instructions:

Equimolar amounts of corresponding chloroquinoxaline derivative and sodium azide are stirred in absolute N,N-dimethylformamide (about 10 ml per mmol of chlorine compound) at 130° C. until the reaction is completed. For working-up, the reaction batch is added to dilute hydrochloric acid (about 100 ml per mmol, the crystalline accumulating reaction product is isolated, washed with water as well as petroleum ether and dried until a constant weight is reached.

Analytically pure product is obtained by recrystallization from a suitable solvent.

### II.3.A. Synthesis of 4-[3(5)-Chloro-1H-pyrazol-5(3)-yl]tetrazolo[1,5-a]quinoxaline (A Compound of General Formula (2) with R1=R2=R3=R4=H, X=Cl)

| Reaction time: | 5 hours | | | |
|---|---|---|---|---|
| Yield: | 86%(light yellow crystals) | | | |
| Summation formula: | C₁₁H₆ClN₇(271.67) | | | |
| Melting point: | 257-258° C.(from ethyl acetate) | | | |
| Elementary analysis: | | C | H | N |
| | Cld. | 48.63% | 2.23% | 36.09% |
| | Fnd. | 48.45% | 2.17% | 35.98% |

| | |
|---|---|
| ¹H-NMR (CDCl₃): | 14.49(s, 1H, NH), 8.65-8.60(m, |
| | 1H), 8.31-8.27(m, 1H), 8.06-7.91(m, |
| | 2H)(H6, H7, H8, H9), 7.62(s, 1H, pyrazole H4) |

The structural modifications that are described in Sections III and IV below can also be used in the N-isosteres of general formula (2) according to the invention.

III. Process for the Production of Compounds of Types (i) and (2) While Varying Substituent R5:

In addition, the invention relates to new parazolyl-substituted [1,2,4]triazolo[4,3-a]quinoxalines, or 1-aza-isosteres, which are characterized in that in the pyrazole ring, there is no longer a free NH function, but rather another substituent, preferably alkyl, allyl, (hetero)aralkyl or acyl, carries one of the two nitrogen atoms. The introduction of this substituent R5 is carried out preferably by subsequent derivatization, for example alkylation, of a compound of general formula (1), in which R5 is equal to hydrogen:

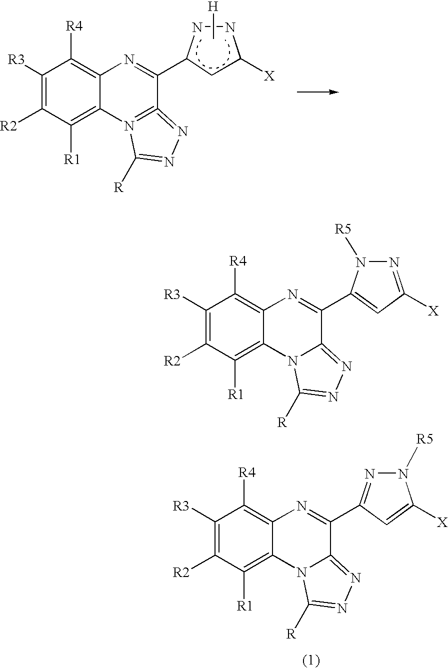

Owing to the chemical nature of the pyrazole, the formation of the R5-position isomers can be expected, by which by selection of the suitable reaction conditions, one of the R5-position isomers should preferably be produced. Optionally-occurring product mixtures can be separated by purification processes that are known to one skilled in the art, such as fractionated crystallization or chromatographic separating processes into the isomer-pure R5-substitution products of general formula (I), as shown above.

Based on some examples, this structural variation is shown, whereby the invention is not to be limited to this example, however; other operating methods that are described for alkylating reactions can also be used.

General Operating Instructions for Alkylation of N-Unsubstituted Derivatives of General Formula (I)

2 equivalent bases (e.g., powdered potassium hydroxide, sodium, sodium hydride, potassium-tert-butanolate) are added to a solution of one equivalent of the corresponding N-unsubstituted derivative of general formula (I) in about 5 ml/mmol of an absolute solvent (for example, dimethyl sulfoxide, tetrahydrofuran, 1,4-dioxane, DMF) under nitrogen atmosphere, after which the mixture is stirred for one hour at room temperature. Then, 2 equivalents of the alkylating agent (preferably alkyl halide or mesylate) are added, after which the reaction mixture is stirred until the reaction is completed at room temperature (TLC monitoring: several drops of the reaction batch are mixed with dilute hydrochloric acid and, extracted with ethyl acetate, mobile solvent: for example, dichloromethane/ethyl acetate=19/1). For working-up, the reaction batch is added in dilute hydrochloric acid (about 100 ml/mmol). The precipitate that is produced is filtered off by suction and washed with water as well as petroleum ether or extracted by extraction with an organic solvent. The dried crude product is then pre-purified by treatment with activated carbon. A subsequent purification/separation of the positional isomers is carried out by chromatography, optionally also by fractionated crystallization.

The structures of these derivatives can be determined unambiguously, for example by means of x-ray structural analysis.

With use of these general operating instructions, the following synthesis examples are performed for the production of compounds of general formula (I):

### Synthesis of 4-(5-Chloro-1H-1-methyl-pyrazol-3-yl)-1-(2-thienylmethyl)-[1,2,4]triazolo[4,3-a]-quinoxaline

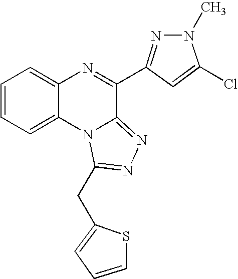

Solvent: Absolute dimethyl sulfoxide

Base: Powdered potassium hydroxide

Reaction time: 1.5 hours

Appearance: Yellow, platy-rhombic crystals

Summation formula: C₁₈H₁₃ClN₆S (380.86)

Melting point: 205-207° C. (from ethyl acetate)

¹H-NMR (CDCl₃): 8.32-8.27 (m, 1H), 8.06-8.01 (m, 1H), 7.66-7.50 (m, 2H) (H6, H7, H8, H9), 7.87 (s, 1H, pyrazole H4), 7.24-7.22 (m, 1H), 6.95-6.90 (m, 1H), 6.81-6.80 (m, 1H) (thiophene H3, H4, H5), 5.11 (s, 2H, CH₂), 4.08 (s, 3H, CH₃)

### Synthesis of 4-[3(5)-Chloro-1-methyl-1H-pyrazol-5(3)-yl]-1-(3-phenyl]propyl)-[1,2,4]triazolo[4,3-a]-quinoxaline as Well as Separation into its Positional Isomers

Solvent: Absolute THF

Base: Powdered potassium hydroxide

Isomer separation: Column chromatography:
Stationary phase: silica gel
Mobile phase: 1,4-dioxane+diisopropyl ether=1+1)

### 4-[5-Chloro-1-methyl-pyrazol-3-yl]-isomer

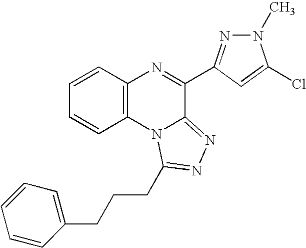

Appearance: Colorless crystals

Purification: Recrystallization from EA

Summation formula: C₂₂H₁₉ClN₆

(402.86)

Yield: 0.145 g (32%)

Melting point: 179-180° C.

IR [cm⁻¹]: 3432, 3158, 2953 (NH)

¹H-NMR (CDCl₃): 8.29 (d, J=7.6 Hz, 1H), 7.86-7.80 (m, 1H), 7.65-7.48 (m, 2H)) (H6, H7, H8, H9), 7.86 (s, 1H, pyrazole-H), 7;38-7.20 (m, 5H, phenyl-H), 4.07 (s, 3H, CH₃), 3.50 (t, J=7.5 Hz, 2H, CH₂), 2.93 (t, J=7.0 Hz, 2H, CH₂), 2.48-2.33 (m, 2H, CH₂)

### 4-[3-Chloro-1-methyl-pyrazol-5-yl]-isomer

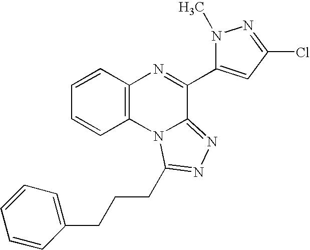

Appearance: Colorless crystals

Purification: Recrystallization from EA

Summation formula: C₂₂H₁₉ClN₆ (402.86)

Yield: 0.260 g (58%)

Melting point: 158-160° C.

IR [cm⁻¹]: 3432 (NH)

¹H-NMR (CDCl₃): 8.12 (dd, J=8.1 Hz, J=1.5 Hz, 1H), 7.83 (dd, J=8.1 Hz, J=1.5 Hz, 1H), 7.69-7.52 (m, 2H) (H6, H7, H8, H9), 7.88 (s, 1H, pyrazole-H), 7.38-7.21 (m, 5H, phenyl-H), 4.40 (s, 3H, CH₃), 3.49 (t, J=7.6 Hz, 2H, CH₂), 2.93 (t, J=7.2 Hz, 2H, CH₂)

A derivatization on the nitrogen atom of the pyrazole ring can take place not only in the stage of the tri- or tetrazoloquinoxazoline derivative, but also in an earlier reaction stage. For the compounds in question, the latter can be performed starting from compounds of type (D), (E), (F) or (G). The subsequent synthesis diagram combines the different possibilities. A process for introducing an acyl group is described in the literature for (E) with R1 to R4 equal to hydrogen (B. Matuszczak et al., J. Heterocyclic Chem. 35: 113-115, 1998). Starting from compounds of type (D), it is preferred to incorporate the derivatization on a compound that has a lactam protective group on the nitrogen. 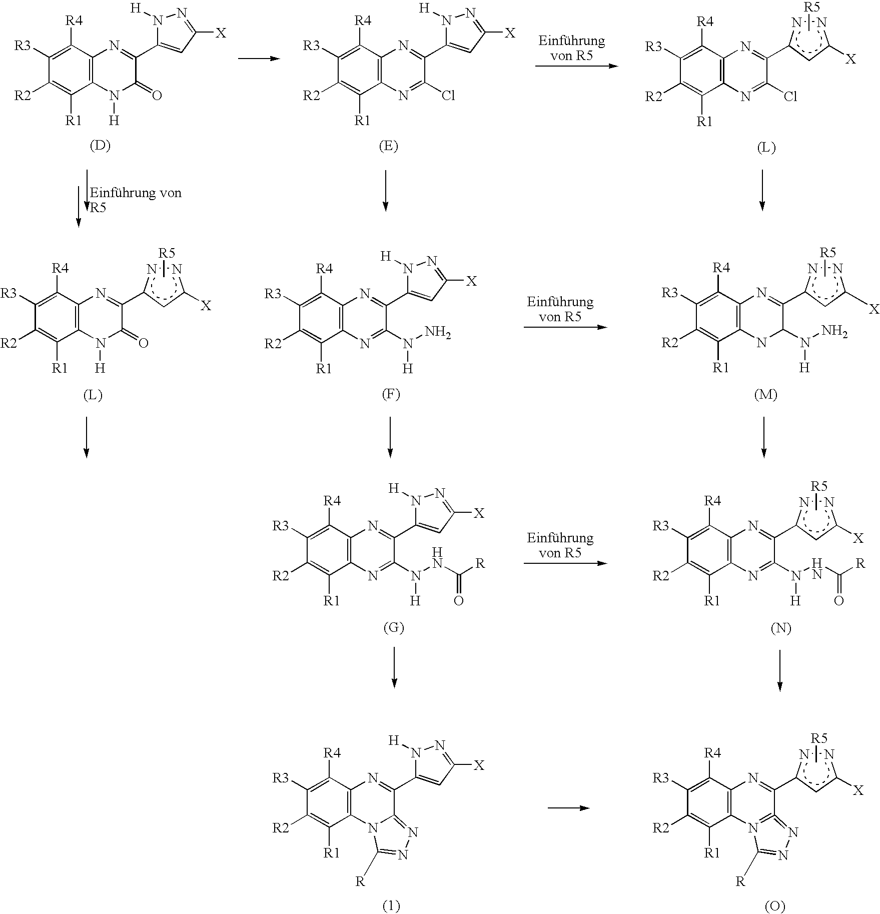
[Key:]
Einführung von R5=Introduction of R5

The introduction of substituents R5, starting from E, is to be described in more detail below based on an example. The invention in question is not limited thereto, but can optionally also easily be transferred to other substituents by slight modification of the reaction conditions. By variation of the conditions, especially by solvent, base, alkylating agent, temperature and optionally catalyst, the ratio of the two N-isomers can be varied and thus in the individual case also be shifted in favor of the desired isomer. 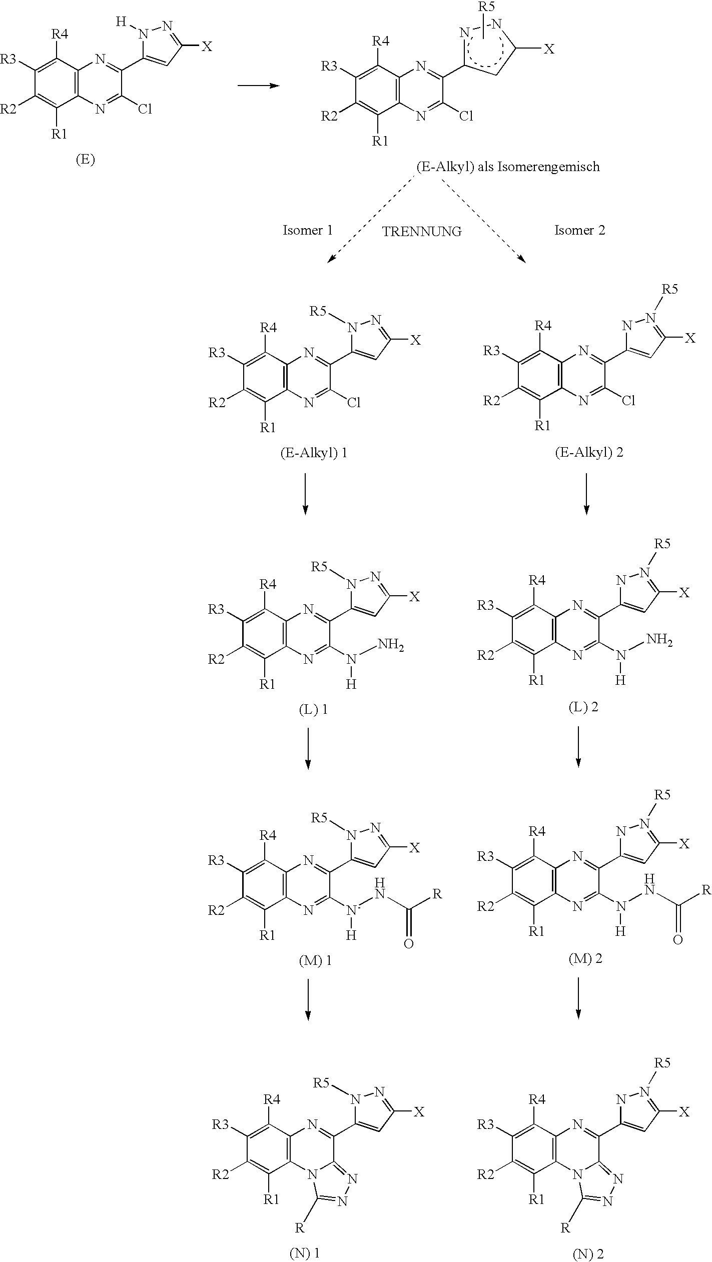
[Key:]
(E-Alkyl) als Isomerengemisch=(E-Alkyl) as isomer mixture
TRENNUNG=SEPARATION

### Alkylation of a 2-Chloro-3-[3(5)-chloro-1H-pyrazol-5(3)-yl]quinoxaline for the Production of Compounds of the (E-Alkyl) Type

2 equivalents of a base (for example, powdered potassium hydroxide, sodium, sodium hydride, potassium-tert-butanolate) and, if necessary, catalytic amounts of an inorganic iodide, are added to a solution or suspension of one equivalent of a chloroquinoxaline of type (E) in an inert solvent (for example THF, DMF, 1,4-dioxane, DMSO; in each case preferably absolute solvent) under nitrogen atmosphere. The mixture is stirred for 1 hour at room temperature. When a suspension is present, an ultrasound treatment can have an advantageous effect on the deprotonation and thus on the further course of the reaction. Then, 2 equivalents of the corresponding alkylating agent, such as alkyl halides or mesylates, are added, after which the reaction mixture is stirred until the reaction is completed (necessary reaction temperature: room temperature up to the boiling range of the solvent that is used; the course of the reaction is tracked by mean of TLC, a sample of the reaction batch is mixed with dilute hydrochloric acid for this purpose and extracted with ethyl acetate; mobile solvent: suitable solvent or mixture). If necessary, i.e., in the case of reaction that is incomplete or proceeds too slowly, more base and/or alkylating agent can be added later. For working-up, the reaction mixture is added to dilute hydrochloric acid, and the reaction product is isolated. This is carried out by separating the precipitate that is produced or by extraction with an organic solvent, after which it is then washed with water. The separation of the isomers as well as their purification is carried out by means of chromatography and/or recrystallization.

The general operating instructions are explained in more detail based on the following examples:

### A) Synthesis of 2-Chloro-3-[3(5)-chloro-1-ethyl-1H-pyrazol-5(3)-yl]-quinoxaline with R5=Ethyl

Base: Powdered potassium hydroxide

Alkylating agent: Ethyl iodide

Solvent: Absolute THF

Isomer separation: Column chromatography:
Stationary phase: silica gel
Mobile phase: dichloromethane+ether=49+1)

Summation formula: C₁₃H₁₀Cl₂N₄ (293.16)

1^{st} Isomer:

Appearance: Colorless crystals

Melting point: 149° C. (from diisopropyl ether)

¹H-NMR (CDCl₃): 8.15-7.84 (m, 4H, H6, H7, H8, H9), 6.78 (s, 1H, pyrazole H4), 4.32 (q, J=7.2 Hz, 2H, CH₂), 1.49 (t, J=7.2 Hz, 3H, CH₃)

2^{nd} Isomer:

Appearance: Yellow, platy-rhombic crystals

Melting point: 126° C. (from diisopropyl ether)

¹H-NMR (CDCl₃): 8.23-8.18 (m, 1H), 8.06-8.01 (m, 1H), 7.82-7.76 (m, 2H) (H6, H7, H8, H9), 7.04 (s, 1H, pyrazole H4), 4;38 (q, J=7:2 Hz, 2H, CH₂), 1.54 (t, J=7.2 Hz, 3H, CH₃)

### B) Synthesis of 2-Chloro-3-[3(5)-chloro-1-(2-methoxyethyl)-1H-pyrazol-5(3)-yl]-quinoxaline with R5=methoxyethyl

Base: Powdered potassium hydroxide

Alkylating agent: Methanesulfonic acid-(2-methoxyethyl)ester

Catalyst: Potassium iodide

Solvent: Absolute 1,4-dioxane

Isomer separation: Column chromatography:
Stationary phase: Silica gel
Mobile phase: Dichloromethane+diisopropyl ether=1+1)

Summation formula: C₁₄H₁₂Cl₂N₄O (323.18)

1^{st} Isomer:

Appearance: Colorless crystals

Melting point: 89-90° C.

¹H-NMR (CDCl₃): 8.16-8.02 (m, 2H), 7.91-7.79 (m, 2H) (H6, H7, H8, H9), 6.75 (s, 1H, pyrazole H4), 4.53 (t, J=5.5 Hz, 2H, CH₂), 3.65 (t, J=5.5 Hz, 2H, CH₂), 3.12 (s, 3H, CH₃)

2^{nd} Isomer:

Appearance: Colorless needles

Melting point: 125° C.

¹H-NMR (CDCl₃): 8.22-8.16 (m, 1H), 8.06-8.01 (m, 1H), 7.83-7.74 (m, 2H) (H6, H7, H8, H9), 7.04 (s, 1H, pyrazole H4), 4.48 (t, J=5.8 Hz, 2H, CH₂), 3.88 (t, J=5.8 Hz, 2H, CH₂), 3.76 (s, 3H, CH₃)

Production of Compounds of Type L:

For reaction, a suspension of the corresponding isomer-pure substituted 2-chloroquinoxaline (E-alkyl) in hydrazine hydrate (about 10 ml/mmol) is refluxed until the reaction is completed, whereby optionally several ml of a high-boiling solvent, such as, for example, 1,4-dioxane, is added (reaction monitoring: a sample of the reaction batch is extracted with ethyl acetate and monitored with TLC, mobile solvent: suitable solvent or mixture). For working-up, the reaction mixture is added to ice water, and the reaction product is isolated by separation of the precipitate that is produced or by extraction with an organic solvent, and it is washed with water. Analytically pure products are obtained from a suitable solvent by recrystallization. If necessary, the crude product is purified by chromatography in advance.

These general operating instructions are explained in more detail based on the following synthesis example:

### Synthesis of 3-[3(5)-Chloro-1-ethyl-1H-pyrazol-5(3)-yl]-2-hydrazinoquinoxaline

Summation formula: C₁₃H₁₃ClN₆ (288.74)

1^{st} Isomer:

Appearance: Yellowish crystals

Yield: 83%

Melting point: 141-143° C.

¹H-NMR (CDCl₃): 7.96-7.91 (m, 1H), 7.83-7.79 (m, 1H), 7.72-7.64 (m, 1H), 7.54-7.45 (m, 1H) (H6, H7, H8, H9), 6.62 (s, 1H, pyrazole H4), 4.33 (q, J=7.2 Hz, 2H, CH₂), 4.18 (s br 2H, NH₂), 1.47 (t, J=7.2 Hz, 3H, CH₃)

2^{nd} Isomer:

Appearance: Yellowish crystals

Yield: 96%

Melting point: 202-204° C.

¹H-NMR (CDCl₃): 9.29 (s, 1H, NH), 7.92-7.87 (m, 1H), 7.75-7.70 (m, 1H), 7.61-7.53 (m, 1H), 7.53-7.35 (m, 1H) (H6, H7, H8, H9), 7.17 (s, 1H, pyrazole H4), 4.34-4.23 (m, 4H, NH₂, CH₂), 1.52 (t, J=7.2 Hz, 3H, CH₃)

Production of Compounds of Type M and Type N:

I) Production of Derivatives with Hydrogen Atom in 1-Position by Reaction of the Compounds of Type L with Orthoformate

The production is carried out by reaction of the isomer-pure hydrazine of type L according to general operating instructions (see Section II.2). In this connection, the following synthesis examples are indicated:

### Synthesis of 4-[3(5)-Chloro-1-ethyl-1H-pyrazol-5(3)-yl]-[1,2,4]triazolo[4,3-a]-quinoxaline

Summation formula: C₁₄H₁₁ClN₆ (298.74)

1^{st} Isomer:

Appearance: Colorless crystals

Melting point: 282-284° C. (from THF)

¹H-NMR (CDCl₃): 9.36 (s, 1H, CH), 8.19-8.14 (m, 1H), 8.01-7.97 (m, 2H), 7.82-7.69 (m, 2H) (H6, H7, H8, H9, pyrazole H4), 4.32 (q, J=7.2 Hz, 2H, CH₂), 1.49 (t, J=7.2 Hz, 3H, CH₃)

2^{nd} Isomer:

Appearance: Colorless crystals

Melting point: 191-192° C. (from ethyl acetate)

¹H-NMR (CDCl₃): 9.36 (s, 1H, CH), 8.37-8.31 (m, 1H), 7.99-7.93 (m, 1H), 7;75-7.65 (m, 2H), (H6, H7, H8, H9), 7.88 (s, 1H, pyrazole H4), 4.46 (q, J=7.3 Hz, 2H, CH₂), 1.57 (t, J=7.3 Hz, 3H, CH₃)

II) Reaction of the Compounds of Type L with Acid Chlorides

Starting from compounds of type L, substituted [1,2,4]triazolo[4,3-a]quinoxalines can be produced according to the methods already previously described, i.e., acylation and subsequent cyclization (see production of compounds of type G-1 and type (1)).

The production is carried out by reaction of the isomer-pure hydrazine of type L according to general operating instructions (see Section II.2) or by a single-pot process, i.e., the hydrazide is not isolated, but rather also converted directly into the tricyclic compound. Although this process is described only on this spot, it is also suitable for the production of pyrazolyl-unsubstituted derivatives. In this connection see, for example, the following:

### Synthesis of 4-[3(5)-Chloro-1-ethyl-1H-pyrazol-5(3)-yl]-1-(3-phenylpropyl)-[1,2,4]triazolo[4,3-a]-quinoxaline

Summation formula: C₂₃H₂₁ClN₆ (416.92)

1^{st} Isomer:

Production in a Single-Pot Process with Use of Acetonitrile as a Solvent

Appearance: Colorless crystals

Melting point: 175-176° C. (from ethyl acetate)

¹H-NMR (CDCl₃): 8.13-8.08 (m, 1H), 7.89 (s, 1H), 7.86-7.81 (m, 1H), 7.69-7.53 (m, 2H), (H6, H7, H8, H9, pyrazole H4), 7.38-7.21 (m, 5H, phenyl-H), 4;85 (q, J=7.2 Hz, 2H, CH₂), 3.49 (t, J=7.8 Hz, 2H, CH₂), 2.94 (t, J=7.1 Hz, 2H, CH₂), 2.48-2.33 (m, 2H, CH₂), 1.55 (t, J=7.2 Hz, 3H, CH₃)

2^{nd} Isomer:

Production in 2 Stages, Namely Acylation and Cyclization with Use of Acetonitrile as a Solvent

Appearance: Colorless needles

Melting point: 146-147° C. (from diisopropyl ether)

¹H-NMR (CDCl₃): 8.33-8.29 (m, 1H), 7.86-7.180 (m, 2H), 7.66-7.48 (m, 2H)) (H6, H7, H8, H9, pyrazole H4), 7.38-7.20 (m, 5H, phenyl-H), 4.44 (q, J=7.3 Hz, 2H, CH₂), 3.50 (t, J=7.7 Hz, 2H, CH₂), 2.94 (t, J=7.1 Hz, 2H, CH₂), 2.48-2.34 (m, 2H, CH₂), 1.55 (t, J=7.3 Hz, 3H, CH₃)

IV. Process for the Production of Compounds of Type (1) or Type (2) While Varying Radical X:

The invention relates to additional pyrazolyl-substituted [1,2,4]triazolo[4,3-a]quinoxaline or tetrazolo derivatives, in which the chlorine atom that is bonded to the pyrazole ring is replaced by other halogens or by hydrogen. The dehalogenation can be carried out, for example, reductively, i.e., by reaction in hydrogen atmosphere or with a hydrogen deliverer such as ammonium formate in the presence of a suitable catalyst (for example, palladium on activated carbon (B. Matuszczak, K. Mereiter, ‘Synthesis in the Series of Pyrazolyl-Substituted Quinoxalines,’ Heterocycles, 45, 2449-2462 (1997)). This dehalogenation reaction is enhanced by reaction at elevated temperature: As solvents, primarily alcohols, tetrahydrofuran as well as 1,4-dioxane are suitable.

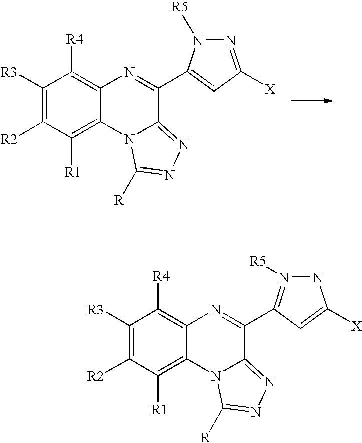

The compounds in their salts, in particular for pharmaceutical use, optionally can be converted into their physiologically compatible salts with an inorganic or organic acid. As an acid, for example, succinic acid, hydrogen bromide, acetic acid, fumaric acid, maleic acid, methanesulfonic acid, lactic acid, phosphoric acid, hydrochloric acid, sulfuric acid, tartaric acid or citric acid is considered for this purpose. Also, mixtures of the previously mentioned acids can be used.

If an acid function is present in the molecule, this compound can also be converted into a physiologically compatible salt for pharmaceutical use. The counterions can have both an inorganic and an organic nature. In this case, ions consist of alkali metals, alkaline-earth metals, ammonium as well as ammonium derivatives, in which instead of hydrogen, one to four organic substituents, preferably alkyl radicals, are present.

In addition, the compounds can also be present in the form of physiologically compatible solvates, especially hydrates.

In the section below, several results of the biological testing of selected representatives are listed. The compound number corresponds to the number that is described in the synthetic portion.

### Biological Tests of the Compounds According to the Invention According to General Formula (1) or (2) as Well as Their Evaluation

The data of the biological tests that are cited below results from radioligand-binding assays on A₁, A_{2A}, A_{2B} and A₃ adenosine receptors (ARs).

The following selective radioligands are used in the biological tests:
[³H]CCPA (for A₁ ARs) [Klotz et al., Naunyn-Schmiedeberg's Arch. Pharmacol., 340, 679 (1989)]
[³]MSX-2 (for A_{2A} ARs) [Muiller et al., Eur. J. Pharm. Sci., 10, 259 (2000)]
[³H]ZM241385 (for A_{2B} ARs_[X.-D. Ji, K. A. Jacobson, Drug Des. Discov., 16, 217 (1999)]
[³H]PSB-11 (new antagonistic radioligand for human A₃ ARs) [M. Dieckmann, M. Thorand, V. Ozola, C. E. Muiller, in Vorbereitung [Preparation] (2001)].
[¹²⁵I]AB-MECA (for rat-A₃ ARs) [Olah et al., Mol. Pharmacol., 45, 978 (1994)].
   Description of the Assays That are Used for Testing Substances
   Production of Tissue Preparations Made from Rat Brains

For the preparations, deep-frozen rat brains of the Pel Freez Company (Rogers, Ark., USA) are used. The rat brains are thawed in 0.32 M saccharose solution, the cortex is cut off, and the striata are prepared outside.

The cell decomposing of the cerebral cortex is carried out with a glass-Teflon homogenizer (stages 5-6, 10 seconds) in 0.32 M saccharose solution. The membranes are purified over various centrifuging steps. With centrifuging at 600 g, 4° C., for 10 minutes, a large amount of cell debris is separated via the P1 fraction, and the supernatant (P2 fraction) is further worked up: in the subsequent three centrifuging steps (35,000 g; 4° C.; 60 minutes), a pellet is produced that is resuspended in 50 mmol of TRIS buffer, pH 7.4 with the Ultraturrax (stages 3-4, 3 seconds); the respective supernatant is discarded. The striata are added to cold TRIS buffer, homogenized with the Ultraturrax in stages 3 to 4-(3 seconds) and then centrifuged at 35,000 g, 4° C., for 20 minutes. The supernatant is discarded, and the pellet is resuspended in 50 mmol of TRIS buffer (50 mmol, pH 7.4). The washing process is repeated twice more. The membrane preparations are stored at −80° C. and are held for several months. The cerebral cortex membrane preparation is used for A₁-radioligand binding studies, and the striatum is used for A_{2A}-radiologiand binding studies.

Cell Culture:

As cell cultures, for example, CHO cells, which express the human A₁-adenosine receptor, are used.

The nutrient medium, trypsine and PBS (phosphate-buffered common salt solution) are heated in a water bath to 37° C. The medium is suctioned off from the flask that is approximately 80% to 90% confluently covered with CHO cells. PBS buffer is added, and the latter is allowed to act for about 5 minutes. Then, the buffer is suctioned off, and the cells are covered with trypsine/EDTA solution. After about 5 minutes, the cells are detached from the bottom of the flask. The trypsine/EDTA solution is diluted with at least the same amount of medium. With this mixture, the flask bottoms are rinsed two to three times to detach cells that are still adhering.

Then, the suspension is added to a centrifuge glass and centrifuged at 20° C. for 5 minutes at 1,000 g. The supernatant is suctioned off, and the cell pellet is taken up in fresh medium. The cell suspension is dispersed uniformly into 20 to 25 cell culture flasks that are inscribed with 20 to 25 and that are filled with medium. The flasks are lightly shaken to disperse the cells well and then incubated for 2 to 3 days in an incubator (37° C.; 5% CO₂).

The following medium composition is used, for example, for CHO cells:

DMEM F12 with the following additives:
10% fetal calf serum
1% penicillin/streptomycin (finished mixture with 10,000 U of penicillin and 10 mg of streptomycin)
2 mmol of L-glutamine (stock solution: 200 mmol in 0.85% NaCl solution)
0.2 mg/ml of G418 (genticin sulfate)

For CHO cells that express the A_{2A}-adenosine receptor, the operating steps are performed analogously to the previously mentioned A₁-adenosine receptor-CHO culture. 0.5 U/ml of adenosine deaminase is added to the medium.

In addition, A₂₈-adenosine receptor membranes are prepared, whereby, for example, finished prepared membranes of HEK cells, which express the human A_{2B}-receptor in high density (about 4 pmol/mg of protein), were available commercially (Receptor Biology USA via Perkin Elmer Life Sciences, Cologne, Germany).

For CHO cells, which express the A₃-adenosine receptor, the operating steps are also performed analogously to the A₁-adenosine receptor-CHO culture. The medium is used as described above.

Membrane Preparation of Recombinant Human Receptors:

Preparation of recombinant, human A₁-adenosine receptors expressed by CHO cells for A₁-adenosine receptor binding studies:

2 to 3 ml of ice-cold TRIS buffer (pH 7.4; 50 mmol) is added to the small tissue culture dishes. The cells are scraped off from the plates with a rubber scraper and collected in a beaker. The plates are rinsed with another 1 to 2 ml of TRIS buffer (pH 7.4; 50 mmol) and added to a beaker. The cell suspension is dispersed onto centrifuge tubes, homogenized with the homogenizer at the highest level (3 to 4 seconds) and centrifuged at 4° C., 35,000 g, for 20 minutes. The supernatant is discarded, and the pellet is resuspended in TRIS buffer (pH 7.4; 50 mmol) with the glass-Teflon homogenizer at the highest level for 3 to 4 seconds. This suspension is centrifuged again (4° C.; 35,000 g, 20 minutes). The washing process is repeated another time. The pellet that is obtained is resuspended with the least possible TRIS buffer (pH 7.4; 50 mmol) and delivered in aliquots (0.25 ml and 0.5 ml).

Preparation of recombinant human A_{2A}-adenosine receptors expressed in CHO cells for A_{2A}-adenosine receptor binding studies:

2 to 3 ml of ice-cold TRIS buffer (pH 7.4; 50 mmol) is added to the tissue culture dishes. The cells are scraped off from the plates with a rubber-scraper and collected in a beaker. The plates are rinsed with another 1 to 2 ml of TRIS buffer (pH 7.4; 50 mmol). The cell suspension is dispersed to the centrifuge tubes and centrifuged at 4° C., 35,000 g, for 20 minutes. The supernatant is discarded, and the pellet is resuspended in TRIS buffer (pH 7.4; 50 mmol) with the homogenizer at the highest level for 3 to 4 seconds. This suspension is again centrifuged. The washing process is repeated still another time. The pellet that is obtained is resuspended with the least possible TRIS buffer (pH 7.4; 50 mmol) and delivered in aliquots (0.25 ml and 0.5 ml).

Before the tissue is used in an assay, it must again be washed with TRIS buffer (pH 7.4; 50 mmol) for 20 minutes at 4° C. and 35,000 g. The unspecific binding can thus be reduced.

Preparation of Recombinant Human A₃-Adenosine Receptors Expressed in CHO Cells for A₃-Adenosine Receptor Binding Studies

2 to 3 ml of ice-cold TRIS buffer (pH 7.4; 50 mmol) is added to the tissue culture dishes. The cells are scraped off from the plates with a rubber scraper and collected in a beaker. The plates are rinsed with another 1 to 2 ml of TRIS buffer (pH 7.4; 50 mmol). The cell suspension is dispersed into centrifuge tubes and centrifuged at 4° C., 35,000 g, for 20 minutes. The supernatant is discarded, and the pellet is resuspended in TRIS buffer (pH 7.4; 50 mmol) with the homogenizer at the highest level for 3 to 4 seconds. This suspension is again centrifuged. The washing process is repeated another time. The pellet that is obtained is resuspended with the least possible TRIS buffer (pH 7.4; 50 mmol) and delivered in aliquots (0.25 ml and 0.5 ml).

Radioligand-Receptor Binding Studies:

The test substances are dissolved in DMSO to 10 mmol. The final concentration of DMSO in the assay is 2.5% for A₁- and A_{2A}-adenosine receptor assays and 1-2% for A_{2B}- and A₃-assays. Dilution series of the test substances are produced from the stock solution. Five to eight different test concentrations, which comprise 3 orders of magnitude (e.g., 1 nm to 1000 nm), are used.

Radioligand Binding Studies with [³H]CCPA on A₁-Adenosine Receptors:

Contained in 1 ml of total volume are 25 μl of DMSO (total bond) or 25 μl of 2-chloroadenosine (400 μmol in DMSO, unspecific bond) or 25 μl of test substance in DMSO as well as 775 μl of TRIS buffer, 50 mmol, pH 7.4; 100 g of [3H]CCPA (final concentration: 0.5 nmol; K_{D}-value: 0.2 nmol) and 100 μl of rat brain homogenate (70 μg/ml) or a membrane preparation that consists of CHO cells, which express recombinant human A₁-adenosine receptors (30-50 g/ml) in 50 mmol of TRIS buffer, pH 7.4, mixed with 0.12 I.U. ADA. The mixture is incubated for 90 minutes in an oscillating water bath at 23° C. Then, it is filtered off with a Brandel cell-harvester and washed twice more with ice-cooled TRIS buffer, 50 mmol, pH 7.4 (3 ml). The filter plates are conveyed in scintillation vials, doused with 2.5 ml of Ultima Gold Scintillation cocktail and incubated for at least 6 hours before they are measured in a scintillation counter.

Receptor Binding Assay with [³H]MSX-2 on A_{2A}-Adenosine Receptors:

25 μl of DMSO, NECA (800 μmol) or test substance dilutions
775 μl of TRIS buffer (for production, see above)
100 μl of [³H]MSX-2 (final concentration 1 nmol; K_{D} value 8 nmol)
100 μl of rat-striatum-membrane preparation or preparation of CHO cells, which express the human A_{2A} receptor, incubated with 0.12 IE adenosine-deaminase (protein concentration: 25-75 μg/ml)
1000 μl of final volume

The assay is performed according to the instructions of the receptor-binding assay with [³H]CCPA with consideration of the amounts and solutions that are listed above. The incubation time is 30 minutes at 23° C. in an oscillating water bath. The glass fiber filter is introduced 30 minutes before use in 0.5% PEI solution.

Receptor Binding Assay with [³H]ZM241385 Expressed on Recombinant A_{2B}-Adenosine Receptors in HEK Cells:
10 μl of DMSO, NECA (1 mmol) or test substance dilutions
790 μl of 10 mmol HEPES buffer, pH 7.4 (for production, see above)
100 μl of [³H]ZM241385 (final concentration 5 nmol; K_{D}-value 33 nmol)
100 μl of membrane preparation, incubated for at least 15 minutes with 0.12 IE adenosine-deaminase (protein concentration: 40 μg/ml)
1000 μl of final volume

The membranes are thawed and the receptor-binding assays are carried out with [3H]CPPA according to the instructions given below with consideration for the amounts and solutions listed above. The dilution in the assay is 1:100 (to reduce the DMSO content to 1%, since a higher DMSO concentration has a disadvantageous effect). The incubation time is 30 minutes at 23° C. in an oscillating water bath. The glass filter is introduced into 0.5% PEI solution 30 minutes before use.

Receptor Binding Assay with [³H]PSB-11 on Recombinant A₃-Adenosine Receptors Expressed in CHO Cells:

10 μl of DMSO, R-PIA (100 μmol) or test-substance dilutions
350 μl of 50 mmol of TRIS buffer, pH 7.4 (for production, see above)
40 μl of [³H]PSB-11 (final concentration 0.5 nmol; K_{D}-value 2 nmol)
100 μl of membrane preparation (at least 15 minutes) incubated with 0.12 of IE adenosine deaminase (protein concentration: 100 μg/ml)
500 μl of final volume

The assay is performed with [³H]CCPA according to the instructions under the receptor-binding assays with consideration for the above-listed amounts and solutions. The dilution in the assay is 1:50. The DMSO content is 2% here. The incubation at 23° C. in the oscillating water bath lasts for 45 minutes.

[35 S]GTP γ Assay:

10 μl of DMSO, GTPγS or test-substance dilutions
150 μl of TRIS buffer, 50 mmol, pH 7.4 for [³⁵S]GTPγS assay (see above)
20 μl of [³⁵S]GTPγS (final concentration 0.1 to 0.5 mmol; corresponds to 1250 Ci/mmol)
20 μl of membrane preparation, incubated with 0.12 E adenosine-deaminase (protein concentration: 75 μg/ml)
200 μl of final volume

First, solutions of the test substances in DMSO are produced in the required concentrations. The curve consists of 7 to 8 measuring points (measured in each case as triplets), which extend over a concentration range of 6 to 7 powers of ten. After the addition of 50 mmol of TRIS buffer, 50 mmol, pH 7.4, to the radioligand [³⁵S]GTPγS and the membrane preparation, which was mixed with adenosine deaminase, a final volume of 200 μl is achieved. The incubation is carried out for 45 minutes at 25° C. in an oscillating water bath. Then, the reaction is first stopped with 2 ml of cold washing buffer (50 mmol of TRIS, 5 mmol of MgCl₂×2H₂O, pH 7.4), and the suspension is filtered with a GF/B filter with the aid of the harvester. The reagent glasses are rinsed twice with 2 ml each of column washing buffer. The filter plates are conveyed by means of a punched-out plate into the scintillation vial, which is filled with 2 ml of Scintillation cocktail. The vials are shaken well to wet the filter papers completely. After an incubation of at least 3 hours, the radioactivity in the LS counter is determined by a 2-minute measurement. The curves are reproduced three times in each case.

In the assay, solutions of test substances 1:20 are diluted; the DMSO content in the assay is 5% (V/V).

Evaluation of the Radioligand Binding Studies:

The mean value is calculated from three independent experiments. IC₅₀ and K, values are obtained from the Cheng-Prusoff equation, the concentration and the K_{D} value of the radioligand. For calculation, the program GraphPadPrism™, Version 3.0 (GraphPad, San Diego, Calif., USA), was used.

Results of the Biological Study of Selected Compounds:

TABLE 1

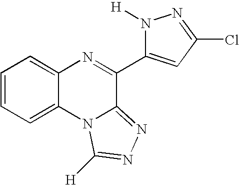

A₁ and A₂ adenosine receptor affinities
[from: B. Matszczak, E. Pekala, C. E. Müller;
Arch. Pharm. Pharm. Med. Chem. 331, 163-169 (1998)]

| | A₁ K₁ [μmol] or | A_{2A} K₁ [pmol] or |
|---|---|---|
| | (% Inhibition @ | (% Inhibition @ |
| Compound | 10 μmol) | 25 μmol) |
| Caffeine | 23.5 ± 3.0 | 32.5 |
| R = —H | >10 | >25 |
| | (35%) | (21%) |
| R = —CH₃ | 7.85 ± 0.95 | 1.43 |
| R = —C₆H₅ | 1.62 ± 0.68 | >25 |
| | | (0%) |
| R =—CH₂C₆H₅ | 0.31 ± 0.09 | >25 |
| | | (6.1% ± 1.5%) |
| R = 2-Furyl | 0.41 ± 0.16 | >25 |
| | | (31% ± 3%) |
| R = 2-Thienyl | 0.71 ± 0.06 | >25 |
| | | (22% ± 13%) |
| R = 2-Thienylmethyl | 0.20 ± 0.01 | >25 |
| | | (21% ± 10%) |

TABLE 2

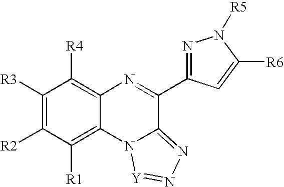

Receptor Affinities of Several New Triazolo- and
Tetrazoloquinoxaline Derivatives
(nt = not tested)

| | | | | A₁ K₁ | A_{2A} K₁ | A_{2B} K₁ | A₃ K₁ |
|---|---|---|---|---|---|---|---|
| | | | | [μmol] or | [μmol] or | [μmol] or | [μmol] or |
| | For | | | (% inhib. @ | (% inhib. @ | (% inhib. @ | (% inhib. @ |
| Y | R1/2/3/4 ≠ H | R5 | R6 | 10 μmol) | 10 μmol) | 1 μmol | 1 μmol) |
| C—CH₂— | R3 = | H | Cl | 0.90 ± | 4.96 ± | >1 | 0.0019 |
| (2- | OCH₃ | | | 0.23 | 1.29 | (0%) | |
| Thienyl) | | | | | | | |
| C—CH₂— | | H | Cl | 0.006 ± | >10 | >1 | >1 |
| CH₂— | | | | 0.001 | (6%) | (18%) | (32%) |
| CH2-Ph | | | | Human: | | | |
| | | | | 2 nmol | | | |
| C—CH₂— | | H | Cl | 0.62 ± | >10 | Nt | Nt |
| O—CH₂-Ph | | | | 0.22 | (3%) | | |
| C—CH— | | H | Cl | 0.10 ± | 1.18 ± | nt | nt |
| (CH₃)—O- | | | | 0.03 | 0.59 | | |
| Ph | | | | | | | |
| C-(3- | | H | Cl | 0.32 ± | 1.26 ± | >1 | >1 |
| Furyl) | | | | 0.07 | 0.18 | (25%) | (31%) |
| C-(3- | | H | Cl | 0.33 ± | 4.10 ± | nt | nt |
| Thienyl) | | | | 0.08 | 1.50 | | |
| C—H | | H | Cl | >10 | >10 | >1 | 0.142 |
| | | | | (35%) | (21%) | (0%) | |
| C—H | R3 = | H | Cl | >10 | >10 | >1 | 0.372 |
| | OCH3 | | | (13%) | (1%) | (8%) | |
| C—CH₂- | | CH₃ | Cl | 0.21 ± | 8.61 ± | >1 | 0.086 |
| (2- | | | | 0.04 | 3.25 | (22%) | |
| Thienyl) | | | | | | | |
| N | | H | Cl | 0.67 ± | 6.13 ± | >1 | >1 |
| | | | | 0.06 | 1.38 | (13%) | (53%) |

Dosages and Forms of Administration

The compounds of general formula (1) or (2) according to the invention can be administered to patients perorally, for example by means of tablets, coated tablets, capsules and drinking solutions; rectally, for example by means of suppositories; by inhalation, for example by inhaling aerosols at defined concentration and size distribution of particles; transdermally, for example by active ingredient-containing patches, rubbing solutions, gels, etc.; transmucosally, for example in terms of a resorption through the mucous membranes of the mouth and nose, whereby the active ingredient in the cavity of the mouth is released by solution in the saliva, or can be introduced into the nose by spray solutions and the like, by means of implanted condensers, which, for example, release the active ingredient by passive osmosis or in a controlled manner by means of mini-pumps or the like; or by an intravenous, intramuscular or subcutaneous injection and intracerebroventricular method.

Typical dosages in the administration of these active ingredients depend on the type of compound that is used and lie in a range of 0.5-100 mg (preferably 1-50 mg) for intravenous administration for an average adult; in the range of 1-1000 mg (preferably 5-500 mg) for peroral administration, and in the range of 0.5-50 mg (preferably 1-20 mg) for transdermal administration.

Owing to the variable administration spectrum, the compounds of general formulas (1) or (2) according to the invention are suitable for the production of pharmaceutical agents for treating diseases in the kidney area, such as in acute renal failure, nephritis, hepatorenal syndrome; for treating the heart, preferably for treating cardiac irregularities, ischemia, myocardial infarction or angina pectoris; for treating the central nervous system (CNS); preferably for treating dementia, Alzheimer's disease, anxiety disorders, epilepsy, Parkinson's disease, stroke, depression, opiate withdrawal or comatose conditions; or for treating the lungs, preferably for treating respiratory diseases, such as asthma, bronchitis and mucoviscidosis; and for treating hypertension, allergic skin diseases, such as urticaria, or inflammations.

In addition, immunostimulants as well as protective agents, which are used in lung transplants, can be produced from compound (1) or (2) according to the invention.

1. Pyrazolyl-substituted [1,2,4-]triazolo[4,3-a]quinoxalines according to formula (1) 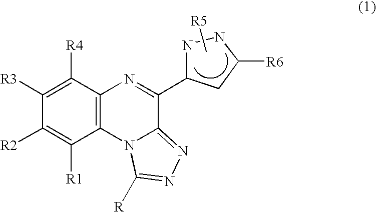
in which R1 to R4 are hydrogen, linear or branched, saturated or unsaturated alkyl radicals, cycloalkyl radicals, which optionally have one or more heteroatoms, aryl or heteroaryl radicals, alkoxy, hydroxy, halogen, amino, nitro, trihalomethyl, carboxy, alkoxycarbonyl or sulfo groups, whereby R1 to R4 are identical or different or can be present as fused aryl or heteroaryl radicals or as correspondingly hydrogenated or partially hydrogenated systems, and in which substituent R is hydrogen or a linear or branched-chain, saturated, and/or unsaturated carbon radical, a cycloalkyl radical, an aryl or heteroaryl radical in substituted or unsubstituted form, whereby substituent R is bonded to the base either directly or via an alkylene group, in which one or more carbon atoms can be replaced by heteroatoms, such as oxygen, sulfur or nitrogen, and in which substituent R5 is hydrogen, C₁-C₈ alkyl, allyl, arylalkyl, (hetero)arylalkyl or acyl, and in which radical R6 is a halogen or hydrogen, excluding compounds with R1 to R5 equal to hydrogen, R6 equal to chlorine and R equal to methyl, phenyl, benzyl, 2-furyl, 2-thienyl or 2-thienylmethyl.

2. Process for the production of pyrazolyl-[1,2,4]triazolo[4,3-a]quinoxalines according to formula (1) of claim 1, characterized in that substituted 1,2 diazines according to formula (C) 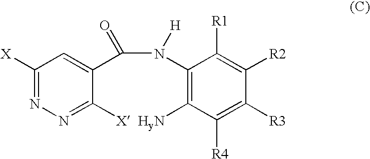
in which X′ is a leaving group and X is halogen or hydrogen, are reacted by ring closure to form pyrazolyl-substituted quinoxalines according to Formula (D) 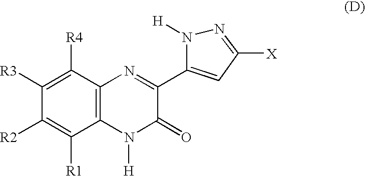
in that then the 2-chloroquinoxaline derivatives according to formula (E) 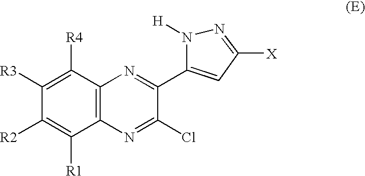
are produced, which are reacted in the corresponding 2-hydrazino derivatives according to formula (F) 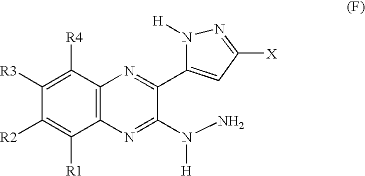
from which the compounds according to Formula (G) 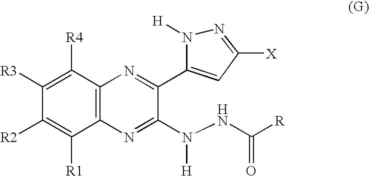
are obtained by acylation, from which the pyrazolyl[1,2,4]triazolo-[4,3-a]quinoxalines according to formula (1) are obtained by ring closure reaction.

3. Process according to claim 2, wherein the ring closure reaction is carried out starting from the corresponding hydrazine without isolating the hydrazide intermediate product.

4. Process according to claim 2, wherein the compounds according to formula (G) are obtained from substituted 2-chloroquinoxalines by reaction with hydrazides.

5. Process for the production of symmetrically substituted pyrazolyl[1,2,4]triazolo[4,3-a]quinoxalines according to claim 2, wherein substituted diazine is obtained according to formula (C) by reaction of o-phenylenediamine according to formula (B), 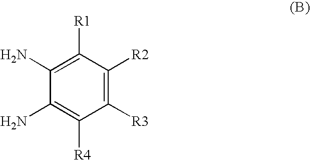
in which radicals R1 to R4 are with activated 3,6-dihalopyridazine-4-carboxylic acid.

6. Process for the production of unsymmetrically substituted pyrazolyl[1,2,4]triazolo[4,3-a]-quinoxalines according to formula (1) of claim 2, wherein substituted diazines according to formula (C), are obtained by N-acylation of the o-nitroaniline derivatives according to formula (H) 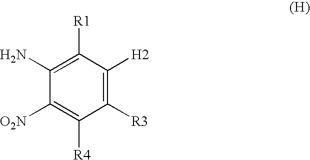
with 3,6-dihalopyridazine-4-carboxylic acid chloride with formation of the compound according to formula (J) 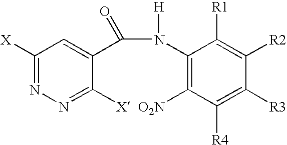
in which the amine group in the compound according to formula (C) is obtained by reduction of the nitro group.

7. Process for the production of unsymmetrically substituted tricyclic compounds according to formula (1) of claim 1, wherein an unsymmetrically substituted phenylenediamine is used with one or more substituents that influence the reactivity, so that at least one of the two amino groups for an acylation reaction is activated or deactivated, by which a selective substitution is carried out on the ring system/on the ring systems.

8. Pyrazolyl-substituted tetrazolo[1,5-a]quinoxalines according to formula (2) 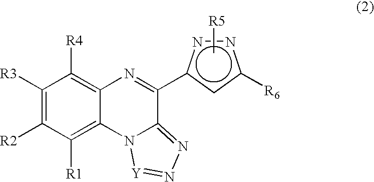
in which R1 to R6 as well as R have the meaning that is indicated in claim 1.

9. Process for the production of compounds according to general formulas (1) or (2) 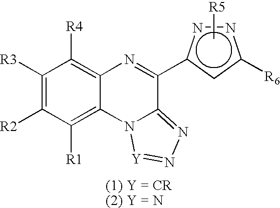
with R5 unequal to hydrogen as well as the separation of the accumulating isomers, wherein either a compound of formula (1) or (2) with R5 equal to hydrogen is alkylated or wherein the introduction of substituent R5 already takes place in the stage of compounds (D), (E), (F) or (G), as indicated in claim 2, whereby the reaction of 2-chloroquinoxaline (E) is preferred.

10. Process for the production of the compound according to formula (2) of claim 9, wherein the compound of formula (E) is produced and reacted with salts of hydrazoic acid.

11. Pyrazolyl[1,2,4]triazolo[4,3-a]quinoxalines according to formula (1) of claim 1 or pyrazolyl-substituted tetrazolo[1,5-a]quinoxalines according to formula (2) that are isolated or in the form of their pharmaceutically compatible salts and solvates as pharmaceutical active ingredients, in particular as adenosine receptor ligands.

12. Pharmaceutical agent, wherein as active ingredient, it contains pyrazolyl-substituted [1,2,4]triazolo[4,3-a]quinoxalines according to formula (1) of claim 1 and/or pyrazolyl-substituted tetrazolo[1,5-a]quinoxalines according to formula (2) and/or their pharmaceutically compatible salts.

13. Pharmaceutical agent according to claim 12, wherein it is present in a form that is suitable for peroral, rectal, inhalational, transdermal, transmucosal, or intracerebroventricular administration.

14. Pharmaceutical agent according to claim 12, wherein it is present in a form of administration that is suitable for implants, infusions or injections.

15. Process for the production of a pharmaceutical agent according to claim 12 for treating diseases in the kidney area, such as for acute renal failure, nephritis, hepatorenal syndrome; for treating the heart, preferably for treating cardiac irregularities, ischemia, myocardial infarction or angina pectoris; for treating the central nervous system (CNS); preferably for treating dementia, Alzheimer's disease, anxiety disorders, epilepsy, Parkinson's disease, stroke, depression, opiate withdrawal or comatose conditions; or for treating the lungs, preferably for treating respiratory diseases, such as asthma, bronchitis and mucoviscidosis.

16. Process for the production of a pharmaceutical agent according to claim 12, wherein as pharmaceutical agents, protective agents, which can be used in lung transplants, are produced.

17. Process for the production of a pharmaceutical agent according to claim 12 for treating hypertension, allergic skin diseases, such as urticaria, or inflammations.

18. Process for the production of a pharmaceutical agent according to claim 12, wherein an immunostimulant is produced as a pharmaceutical agent.
